(19) United States
(12) Patent Application Publication
(10) Pub. No.: US 2016/0272959 A1
(43) Pub. Date: Sep. 22, 2016

(54) VARIANTS OF CELLOBIOHYDROLASES

(71) Applicant: DANISCO US INC., Palo Alto, CA (US)

(72) Inventors: Richard R. BOTT, Burlingame, CA (US); Maria FOUKARAKI, Rotterdam (NL); Ronaldus Wilhelmus HOMMES, Haarlem (NL); Thijs KAPER, Half Moon Bay, CA (US); Bradley R. KELEMEN, Menlo Park, CA (US); Slavko KRALJ, Oegstgeest (NL); Igor NIKOLAEV, Noordwijk (NL); Mats SANDGREN, Uppsala (SE); Johannes Franciscus Thomas VAN LIESHOUT, Utrecht (NL); SANDER VAN STIGT THANS, Zevenbergen (NL)

(73) Assignee: Danisco US Inc., Palo Alto, CA (US)

(21) Appl. No.: 14/650,289

(22) PCT Filed: Dec. 10, 2013
(86) PCT No.: PCT/US2013/074020

§ 371 Date: Jun. 5, 2015

Related U.S. Application Data
(60) Provisional application No. 61/736,344, filed on Dec. 12, 2012.

(51) Int. Cl. C12N 9/42; C12P 19/02; C12P 19/14

(52) U.S. Cl. CPC C12N 9/2437; C12P 19/14; C12P 19/02; C12Y 302/01099

(57) ABSTRACT

Disclosed are a number of homologs and variants of Hypocrea jecorina Cel7A (formerly Trichoderma reesei cellobiohydrolase I or CBH1), nucleic acids encoding the same and methods for producing the same. The homologs and variant cellulases have the amino acid sequence of a glycosyl hydrolase of family 7A wherein one or more amino acid residues are substituted and/or deleted.

### CROSS REFERENCE TO RELATED APPLICATIONS

This application claims benefit of priority from U.S. provisional patent application Ser. No. 61/736,344 filed on 12 Dec. 2012, and is incorporated herein by reference in its entirety.

### GOVERNMENT RIGHTS

This invention was made with government support under grant number DE-FC36-08G018078 awarded by the U.S. Department of Energy. The government has certain rights in this invention.

### FIELD OF THE INVENTION

The present disclosure generally relates to glycoside hydrolase enzyme variants, particularly variants of cellobiohydrolase (CBH). Nucleic acids encoding the CBH variants, compositions including the CBH variants, methods of producing the CBH variants, and methods of using the variants are also described.

### BACKGROUND OF THE INVENTION

Cellulose and hemicellulose are the most abundant plant materials produced by photosynthesis. They can be degraded and used as an energy source by numerous microorganisms, including bacteria, yeast and fungi, that produce extracellular enzymes capable of hydrolysis of the polymeric substrates to monomeric sugars (Aro et al., 2001). As the limits of non-renewable resources approach, the potential of cellulose to become a major renewable energy resource is enormous (Krishna et al., 2001). The effective utilization of cellulose through biological processes is one approach to overcoming the shortage of foods, feeds, and fuels (Ohmiya et al., 1997).

Cellulases are enzymes that hydrolyze cellulose (beta-1,4-glucan or beta D-glucosidic linkages) resulting in the formation of glucose, cellobiose, cellooligosaccharides, and the like. Cellulases have been traditionally divided into three major classes: endoglucanases (EC 3.2.1.4) (“EG”), exoglucanases or cellobiohydrolases (EC 3.2.1.91) (“CBH”) and beta-glucosidases ([beta]-D-glucoside glucohydrolase; EC 3.2.1.21) (“BG”). (Knowles et al., 1987; Shulein, 1988). Endoglucanases act mainly on the amorphous parts of the cellulose fiber, whereas cellobiohydrolases are also able to degrade crystalline cellulose (Nevalainen and Penttila, 1995). Thus, the presence of a cellobiohydrolase in a cellulase system is required for efficient solubilization of crystalline cellulose (Suurnakki, et al. 2000). Beta-glucosidase acts to liberate D-glucose units from cellobiose, cello-oligosaccharides, and other glucosides (Freer, 1993).

Cellulases are known to be produced by a large number of bacteria, yeast and fungi. Certain fungi produce a complete cellulase system capable of degrading crystalline forms of cellulose, such that the cellulases are readily produced in large quantities via fermentation. Filamentous fungi play a special role since many yeast, such as Saccharomyces cerevisiae, lack the ability to hydrolyze cellulose. (See, e.g., Aro et al., 2001; Aubert et al., 1988; Wood et al., 1988, and Coughlan, et al.)

The fungal cellulase classifications of CBH, EG and BG can be further expanded to include multiple components within each classification. For example, multiple CBHs, EGs and BGs have been isolated from a variety of fungal sources including Trichoderma reesei which contains known genes for 2 CBHs, i.e., CBH I and CBH II, at least 8 EGs, i.e., EG I, EG II, EG III, EGIV, EGV, EGVI, EGVII and EGVIII, and at least 5 BGs, i.e., BG1, BG2, BG3, BG4 and BG5.

In order to efficiently convert crystalline cellulose to glucose the complete cellulase system comprising components from each of the CBH, EG and BG classifications is required, with isolated components less effective in hydrolyzing crystalline cellulose (Filho et al., 1996). A synergistic relationship has been observed amongst cellulase components from different classifications. In particular, the EG-type cellulases and CBH-type cellulases synergistically interact to more efficiently degrade cellulose. (See, e.g., Wood, 1985.)

Cellulases are known in the art to be useful in the treatment of textiles for the purposes of enhancing the cleaning ability of detergent compositions, for use as a softening agent, for improving the feel and appearance of cotton fabrics, and the like (Kumar et al., 1997).

Cellulase-containing detergent compositions with improved cleaning performance (U.S. Pat. No. 4,435,307; GB App. Nos. 2,095,275 and 2,094,826) and for use in the treatment of fabric to improve the feel and appearance of the textile (U.S. Pat. Nos. 5,648,263, 5,691,178, and 5,776,757; GB App. No. 1,358,599; The Shizuoka Prefectural Hammamatsu Textile Industrial Research Institute Report, Vol. 24, pp. 54-61, 1986), have been described.

Cellulases are further known in the art to be useful in the conversion of cellulosic feedstocks into ethanol. This process has a number of advantages, including the ready availability of large amounts of feedstock that is otherwise discarded (e.g., burning or land filling the feedstock). Other materials that consist primarily of cellulose, hemicellulose, and lignin, e.g., wood, herbaceous crops, and agricultural or municipal waste, have been considered for use as feedstock in ethanol production.

It would be an advantage in the art to provide cellobiohydrolase (CBH) variants with improved properties for converting cellulosic materials to monosaccharides, disaccharides, and polysaccharides. Improved properties of the variant CBH include, but are not limited to: altered temperature-dependent activity profiles, thermostability, pH activity, pH stability, substrate specificity, product specificity, and chemical stability.

### BRIEF SUMMARY OF THE INVENTION

The present disclosure describes isolated cellobiohydrolase (CBH) enzymes having cellulase activity, nucleic acids encoding such CBH enzymes, host cells containing CBH enzyme-encoding polynucleotides (e.g., host cells that express the CBH enzymes), compositions containing the CBH enzyme, and methods for producing and using the same.

As such, aspects of the present invention provide variant CBH enzymes having improvements over a wild type CBH enzyme, where the variants are significantly improved for one or more characteristic selected from: increased melting temperature (Tm), performance in a PASO Hydrolysis Assay, performance in a Whole Hydrolysate PCS (whPCS) Assay, and performance in a Dilute Ammonia Corn Stover (daCS) Assay. In certain embodiments, the CBH variant has at least two of the improved characteristics, at least three of the improved characteristics, or all of the improved characteristics.

Aspects of the present invention provide an isolated variant of a parent cellobiohydrolase (CBH) enzyme as set forth below, where any indicated CBH amino acid position corresponds to the amino acid sequence in SEQ ID NO:3:

1. An CBH variant where the variant has cellulase activity, has at least 80% sequence identity to SEQ ID NO:3, and has significantly improved performance in a Phosphoric Acid Swollen Cellulose (PASO) Assay.

2. The CBH variant of 1, where the variant comprises an amino acid substitution selected from the group consisting of: Y247D, N49P, T246V, N200G, and combinations thereof.

3. The CBH variant of 2, where the variant comprises a Y247D substitution.

4. The CBH variant of 2 or 3, where the variant comprises a N49P substitution.

5. The CBH variant of 2, 3 or 4, where the variant comprises a T246V substitution.

6. The CBH variant of 2, 3, 4 or 5, where the variant comprises a N200G substitution.

7. The CBH variant of any one of 2 to 6, where the variant further comprises an amino acid substitution selected from the group consisting of: F418M, T246S, T255V, and combinations thereof.

8. The CBH variant of any one of 2 to 7, where the variant further comprises an amino acid substitution selected from the group consisting of: D241N, G234D, P194V, T255I, T255K, T255R, and combinations thereof.

9. The CBH variant of any one of 2 to 8, where the variant further comprises an amino acid substitution selected from the group consisting of: T356L, T246P, T255D, N200R, and combinations thereof.

10. The CBH variant of any one of 2 to 9, where the variant further comprises an amino acid substitution selected from the group consisting of: T255P, S92T, T41I, and combinations thereof.

11. The CBH variant of 3 or 4 where said variant further comprises a T246P substitution.

12. The CBH variant of 3 or 4 where said variant further comprises a T246V substitution.

13. The CBH variant of 3, 4, 5, 11 or 12, where said variant further comprises a N200G substitution.

14. The CBH variant of 3, 4, 5, 11 or 12, where said variant further comprises a N200R substitution.

15. The CBH variant of any one of 3, 4, 5, 6 and 11 to 14, where said variant further comprises a T255V substitution.

16. The CBH variant of any one of 3, 4, 5, 6 and 11 to 14, where said variant further comprises a T255I substitution.

17. The CBH variant of any one of 3, 4, 5, 6 and 11 to 14, where said variant further comprises a T255K substitution.

18. The CBH variant of any one of 3, 4, 5, 6 and 11 to 14, where said variant further comprises a T255R substitution.

19. The CBH variant of any one of 3, 4, 5, 6 and 11 to 14, where said variant further comprises a T255D substitution.

20. The CBH variant of any one of 3, 4, 5, 6 and 11 to 14, where said variant further comprises a T255P substitution.

21. The CBH variant of any one of 3, 4, 5, 6 and 11 to 20, where said variant further comprises a D241N substitution.

22. The CBH variant of any one of 3, 4, 5, 6 and 11 to 21, where said variant further comprises a G234D substitution.

23. The CBH variant of any one of 3, 4, 5, 6 and 11 to 22, where said variant further comprises a P194V substitution.

24. The CBH variant of any one of 3, 4, 5, 6 and 11 to 23, where said variant further comprises a T356L substitution.

25. The CBH variant of any one of 3, 4, 5, 6 and 11 to 24, where said variant further comprises a S92T substitution.

26. The CBH variant of any one of 3, 4, 5, 6 and 11 to 25, where said variant further comprises a T41I substitution.

In certain embodiments, the parent CBH is a fungal cellobiohydrolase 1 (CBH1), e.g., a CBH1 from Hypocrea jecorina, Hypocrea orientalis, Hypocrea schweinitzii, Trichoderma citrinoviride; Trichoderma pseudokoningii; Trichoderma konilangbra, Trichoderma harzanium, Aspergillus aculeatus, Aspergillus niger; Penicillium janthinellum, Humicola grisea, Scytalidium thermophilum, and Podospora anderina (or their respective anamorph, teleomorph or holomorph counterpart forms), e.g., a CBH1 selected from any one of SEQ ID NOs: 3 to 15. In certain embodiments, the parent CBH has at least 90% sequence identity to SEQ ID NO:3, e.g., at least 95% sequence identity.

Aspects of the subject invention include an isolated polynucleotide comprising a polynucleotide sequence encoding a variant of a parent CBH as described herein. The isolated polynucleotide may be present in a vector, e.g., an expression vector or a vector for propagation of the polynucleotide. The vector may be present in a host cell to propagate the vector and/or that expresses the encoded CBH variant as described herein. The host cell can be any cell that finds use in propagation of the CBH variant polynucleotide and/or expression of the encoded CBH variant, e.g., a bacterial cell, a fungal cell, etc. Examples of suitable fungal cell types that can be employed include filamentous fungal cells, e.g., cells of Trichoderma reesei, Trichoderma longibrachiatum, Trichoderma viride, Trichoderma koningii, Trichoderma harzianum, Penicillium, Humicola, Humicola insolens, Humicola grisea, Chrysosporium, Chrysosporium lucknowense, Myceliophthora thermophila, Gliocladium, Aspergillus, Fusarium, Neurospora, Hypocrea, Emericella, Aspergillus niger, Aspergillus awamori, Aspergillus aculeatus, and Aspergillus nidulans. Alternatively, the fungal host cell can be a yeast cell, e.g., Saccharomyces cervisiae, Schizzosaccharomyces pombe, Schwanniomyces occidentalis, Kluveromyces lactus, Candida utilis, Candida albicans, Pichia stipitis, Pichia pastoris, Yarrowia lipolytica, Hansenula polymorpha, Phaffia rhodozyma, Arxula adeninivorans, Debaryomyces hansenii, or Debaryomyces polymorphus.

Aspects of the present invention include methods of producing a variant CBH that includes culturing a host cell that contains a polynucleotide encoding the CBH variant in a suitable culture medium under suitable conditions to express (or produce) the CBH variant from the polynucleotide, e.g., where the polynucleotide encoding the CBH variant is present in an expression vector (i.e., where the CBH variant-encoding polynucleotide is operably linked to a promoter that drives expression of the CBH variant in the host cell). In certain embodiments, the method further includes isolating the produced CBH variant.

Aspects of the present invention also include compositions containing a CBH variant as described herein. Examples of suitable compositions include, but are not limited to detergent compositions, feed additives, and compositions for treating (or hydrolyzing) a cellulosic substrate (e.g., a cellulose containing textile, e.g., denim; a cellulose containing biomass material, e.g., a mixture of lignocellulosic biomass material which has optionally been subject to pre-treatment of pre-hydrolysis processing, etc.). Compositions that include a CBH variant as described herein and a cellulosic substrate represent further aspects of the present invention. CHB variant-containing detergent compositions include laundry detergents and dish detergents, where such detergents may further include additional components, e.g., surfactants. Examples of suitable cellulosic substrates include, but are not limited to: grass, switch grass, cord grass, rye grass, reed canary grass, miscanthus, sugar-processing residues, sugarcane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, wood pulp, recycled wood pulp fiber, paper sludge, sawdust, hardwood, softwood, and combinations thereof.

Aspects of the present invention include methods for hydrolyzing a cellulosic substrate comprising contacting the substrate with a variant CBH as described herein. In certain embodiments, the CBH variant is provided as a cell-free composition, whereas in other embodiments, the CBH variant is provided as a host cell composition in which the host cell expresses the CBH variant. Thus, certain embodiments of the methods for hydrolyzing a cellulosic substrate contacting the substrate with a host cell containing a CBH variant expression vector. In certain embodiments, the method is for converting a lignocellulosic biomass to glucose, where in some of these embodiments, the lignocellulosic biomass is selected, without limitation, from: grass, switch grass, cord grass, rye grass, reed canary grass, miscanthus, sugar-processing residues, sugarcane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, wood pulp, recycled wood pulp fiber, paper sludge, sawdust, hardwood, softwood, and combinations thereof. In certain other embodiments, the cellulosic substrate is a cellulosic-containing textile, e.g., denim, where in some of these embodiments the method is for treating indigo dyed denim (e.g., in a stonewashing process).

Aspects of the present invention include cell culture supernatant compositions that contain a CBH variant as described herein. For example, a cell culture supernatant obtained by culturing a host cell that contains a polynucleotide encoding the CBH variant in a suitable culture medium under suitable conditions to express the CBH variant from the polynucleotide and secrete the CBH variant into the cell culture supernatant. Such a cell culture supernatant can include other proteins and/or enzymes produced by the host cell, including endogenously- and/or exogenously-expressed proteins and/or enzymes. Such supernatant of the culture medium can be used as is, with minimum or no post-production processing, which may typically include filtration to remove cell debris, cell-kill procedures, and/or ultrafiltration or other steps to enrich or concentrate the enzymes therein. Such supernatants are referred to herein as “whole broths” or “whole cellulase broths”.

The CBH variants can be produced by co-expression with one or more other cellulases, and/or one or more hemicellulases. Alternatively, the CBH variants can be produced without other cellulases or hemicellulases. In the latter case, the CBH variant optionally can be physically mixed with one or more other cellulases and/or one or more hemicellulases to form an enzyme composition that is useful for a particular application, e.g., in hydrolyzing lignocellulosic biomass substrates.

Other compositions containing a desired variant cellulase, as well as methods for using such compositions, are also contemplated.

### BRIEF DESCRIPTION OF THE DRAWINGS

FIGS. 1A and 1B show the nucleic acid sequence (top line) (SEQ ID NO:1) and amino acid sequence (bottom line) (SEQ ID NO:3) of the wild type Cel7A (CBH1) from H. jecorina.

FIGS. 2A, 2B, 2C and 2D show the amino acid alignment of the mature form of CBH enzymes derived from Hypocrea jecorina (SEQ ID NO:3), Hypocrea orientalis (SEQ ID NO:4), Hypocrea schweinitzii (SEQ ID NO:5), Trichoderma citrinoviride (SEQ ID NO:6); Trichoderma pseudokoningii (SEQ ID NO:7); Trichoderma konilangbra (SEQ ID NO:8), Trichoderma harzanium (SEQ ID NO:9), Aspergillus aculeatus (SEQ ID NO:10), Aspergillus niger (SEQ ID NO:11); Penicillium janthinellum (SEQ ID NO:12), Humicola grisea (SEQ ID NO:13), Scytalidium thermophilum (SEQ ID NO:14), and Podospora anderina (SEQ ID NO:15). The numbering at the top indicates the amino acid number of the mature form of Hypocrea jecorina. Identical, conserved, and semi-conserved amino acids are indicated with an asterisk (*), colon (:), and period (.), respectively.

FIG. 3 is a schematic representation of the expression vector pTTT-pyrG-cbh1.

FIG. 4 shows CBH substitution variants that display significant changes in melting temperature (ΔTm). ΔTm is on the X axis with each specific variant having significant ΔTm shown at its ΔTm value. The intercept value indicates the model's prediction of ΔTm for a molecule with no substitutions (i.e., wild type).

FIG. 5 shows CBH substitution variants that display significant changes in performance index (ΔPI) in a whPCS assay. ΔPI is on the X axis (labeled “Benefit to whPCS PI”) with each specific variant having significant ΔPI shown at its approximate ΔPI value. The intercept value indicates the model's prediction of ΔPI for a molecule with no substitutions (i.e., wild type).

FIG. 6 shows CBH substitution variants that display significant changes in performance index (ΔPI) in a daCS assay. ΔPI is on the X axis (labeled “Benefit to daCS PI”) with each specific variant having significant ΔPI shown at its approximate ΔPI value. The intercept value indicates the model's prediction of ΔPI for a molecule with no substitutions (i.e., wild type).

FIG. 7 shows CBH substitution variants that display significant changes in performance index (ΔPI) in a PASO assay. ΔPI is on the X axis (labeled “Benefit to daCS PI”) with each specific variant having significant ΔPI shown at its approximate ΔPI value. The intercept value indicates the model's prediction of ΔPI for a molecule with no substitutions (i.e., wild type).

FIGS. 8A, 8B and 8C show the CBH1 amino acid sequence from H. jecorina containing the amino acid substitutions described herein (SEQ ID NO:16). The designation “Xaa” indicates an amino acid position at which more than one substitution can be made. The substitutions at these Xaa sites are indicated at the bottom of FIG. 8C (at positions 200, 246 and 255). Substituted amino acid positions are in bold underline.

### DETAILED DESCRIPTION

The invention will now be described in detail by way of reference only using the following definitions and examples. All patents and publications, including all sequences disclosed within such patents and publications, referred to herein are expressly incorporated by reference.

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 3RD ED., John Wiley and Sons, Ltd., New York (2007), and Hale & Marham, THE HARPER COLLINS DICTIONARY OF BIOLOGY, Harper Perennial, NY (1991) provide one of skill with a general dictionary of many of the terms used in this invention. Although any methods and materials similar or equivalent to those described herein can be used in the practice or testing of the present invention, the preferred methods and materials are described. Numeric ranges are inclusive of the numbers defining the range. Unless otherwise indicated, nucleic acids are written left to right in 5′ to 3′ orientation; amino acid sequences are written left to right in amino to carboxy orientation. Practitioners are particularly directed to Green and Sambrook Molecular Cloning: A Laboratory Manual (Fourth Edition), Cold Spring Harbor Laboratory Press 2012, and Ausubel F M et al., 1993, for definitions and terms of the art. It is to be understood that this invention is not limited to the particular methodology, protocols, and reagents described, as these may vary.

The headings provided herein are not limitations of the various aspects or embodiments of the invention which can be had by reference to the specification as a whole. Accordingly, the terms defined immediately below are more fully defined by reference to the specification as a whole.

All publications cited herein are expressly incorporated herein by reference for the purpose of describing and disclosing compositions and methodologies which might be used in connection with the invention.

### I. DEFINITIONS

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “nucleic acid” encompasses DNA, RNA, heteroduplexes, and synthetic molecules capable of encoding a polypeptide. Nucleic acids may be single stranded or double stranded, and may have chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. As such, the present invention contemplates every possible variant nucleotide sequence encoding CBH or a variant thereof, all of which are possible given the degeneracy of the genetic code. Unless otherwise indicated, nucleic acid sequences are presented in 5′-to-3′ orientation.

“Cellulase” or “cellulase enzyme” means bacterial or fungal exoglucanases or exocellobiohydrolases, and/or endoglucanases, and/or β-glucosidases. These three different types of cellulase enzymes are known to act synergistically to convert cellulose and its derivatives to glucose.

“Cellobiohydrolase” or “CBH” or “CBH enzyme” or “CBH polypeptide” as used herein is defined as a 1,4-D-glucan cellobiohydrolase (E.C. 3.2.1.91) which catalyzes the hydrolysis of 1,4-beta-D-glucosidic linkages in cellulose, cellotetriose, or any beta-1,4-linked glucose containing polymer, releasing cellobiose from the non-reducing ends of the chain. Cellobiohydrolase (CBH) activity is determined for purposes of the present invention according to the procedures described by Lever et al., 1972, Anal. Biochem. 47: 273-279 and variations thereof (see Examples section below), and/or by van Tilbeurgh et al., 1982, FEBS Letters, 149: 152-156.

A “variant” of an enzyme, protein, polypeptide, nucleic acid, or polynucleotide as used herein means that the variant is derived from a parent polypeptide or parent nucleic acid (e.g., native, wildtype or other defined parent polypeptide or nucleic acid) that includes at least one modification or alteration as compared to that parent. Alterations/modifications can include a substitution of an amino acid/nucleic acid residue in the parent for a different amino acid/nucleic acid residue at one or more sites, deletion of an amino acid/nucleic acid residue (or a series of amino acid/nucleic acid residues) in the parent at one or more sites, insertion of an amino acid/nucleic acid residue (or a series of amino acid/nucleic acid residues) in the parent at one or more sites, truncation of amino- and/or carboxy-terminal amino acid sequences or 5′ and or 3′ nucleic acid sequences, and any combination thereof. A variant CBH enzyme (sometimes referred to as a “CBH variant”) according to aspects of the invention retains cellulase activity but may have an altered property in some specific aspect, e.g., an improved property. For example, a variant CBH enzyme may have an altered pH optimum, improved thermostability or oxidative stability, or a combination thereof, but will retain its characteristic cellulase activity.

“Combinatorial variants” are variants comprising two or more mutations, e.g., 2, 3, 4, 5, 6, 7, 8, 9, 10, etc., substitutions, deletions, and/or insertions.

A “parent CBH1 enzyme” or “parent CBH enzyme” or “parent CBH polypeptide” or equivalents thereto as used herein means a polypeptide that in its mature form comprises an amino acid sequence which has at least 80% identity with SEQ ID NO: 3, including amino acid sequences having at least 81%, 82%, 83%, 84%, 85%, 86%, 87%, 88%, 89%, 90%, 91%, 92%, 93%, 94%, 95%, 96%, 97%, 98%, 99%, or 100% identity with SEQ ID NO: 3, which provides the amino acid sequence of the mature form of wild type CBH1 from Hypocrea jecorina. It is noted that the words “parent” and “parental” are used interchangeably in this context. In certain aspects, a parent CBH enzyme comprises the amino acid sequence of any one of SEQ ID NOs: 2 to 8, or an allelic variant thereof, or a fragment thereof that has cellulase activity. In certain embodiments, the parent CBH enzyme is from a filamentous fungus of the subdivision Eumycota or Oomycota. The filamentous fungi are characterized by vegetative mycelium having a cell wall composed of chitin, glucan, chitosan, mannan, and other complex polysaccharides, with vegetative growth by hyphal elongation and carbon catabolism that is obligately aerobic. A filamentous fungal parent cell may be a cell of a species of, but not limited to, Trichoderma, e.g., Trichoderma longibrachiatum, Trichoderma viride, Trichoderma koningii, Trichoderma harzianum; Penicillium sp.; Humicola sp., including Humicola insolens and Humicola grisea; Chrysosporium sp., including C. lucknowense; Myceliophthora sp.; Gliocladium sp.; Aspergillus sp.; Fusarium sp., Neurospora sp., Hypocrea sp., e.g., Hypocrea jecorina, and Emericella sp. As used herein, the term “Trichoderma” or “Trichoderma sp.” refers to any fungal strains which have previously been classified as Trichoderma or are currently classified as Trichoderma.

The term “wild-type” refers to a naturally-occurring polypeptide or nucleic acid sequence, i.e., one that does not include a man-made variation.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous polypeptide will often refer to two or more subsequences that are not found in the same relationship to each other in nature (e.g., a fusion polypeptide).

The term “recombinant” when used with reference, e.g., to a cell, or nucleic acid, polypeptide, or vector, indicates that the cell, nucleic acid, polypeptide or vector, has been modified by the introduction of a heterologous nucleic acid or polypeptide or the alteration of a native nucleic acid or polypeptide, or that the cell is derived from a cell so modified. Thus, for example, recombinant cells express genes that are not found within the native (non-recombinant) form of the cell or express native genes that are otherwise abnormally expressed, under expressed or not expressed at all.

The terms “isolated” or “purified” as used herein refer to a nucleic acid or polynucleotide that is removed from the environment in which it is naturally produced. In general, in an isolated or purified nucleic acid or polypeptide sample, the nucleic acid(s) or polypeptide(s) of interest are present at an increased absolute or relative concentration as compared to the environment in which they are naturally produced.

The term “enriched” when describing a component or material in a composition (e.g., a polypeptide or polynucleotide) means that the component or material is present at a relatively increased concentration in that composition as compared to the starting composition from which the enriched composition was generated. For example, an enriched CBH composition (or sample) is one in which the relative or absolute concentration of CBH is increased as compared to the initial fermentation product from the host organism.

As used herein, the terms “promoter” refers to a nucleic acid sequence that functions to direct transcription of a downstream gene. The promoter will generally be appropriate to the host cell in which the target gene is being expressed. The promoter, together with other transcriptional and translational regulatory nucleic acid sequences (also termed “control sequences”), are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences. A “constitutive” promoter is a promoter that is active under most environmental and developmental conditions. An “inducible” promoter is a promoter that is active under environmental or developmental regulation. An example of an inducible promoter useful in the present invention is the T. reesei (H. jecorina) cbh1 promoter which is deposited in GenBank under Accession Number D86235. In another aspect the promoter is a cbh II or xylanase promoter from H. jecorina. Examples of suitable promoters include the promoter from the A. awamori or A. niger glucoamylase genes (Nunberg, J. H. et al. (1984) Mol. Cell. Biol. 4, 2306-2315; Boel, E. et al. (1984) EMBO J. 3, 1581-1585), the Mucor miehei carboxyl protease gene, the Hypocrea jecorina cellobiohydrolase I gene (Shoemaker, S. P. et al. (1984) European Patent Application No. EPO0137280A1), the A. nidulans trpC gene (Yelton, M. et al. (1984) Proc. Natl. Acad. Sci. USA 81, 1470-1474; Mullaney, E. J. et al. (1985) Mol. Gen. Genet. 199, 37-45) the A. nidulans alcA gene (Lockington, R. A. et al. (1986) Gene 33, 137-149), the A. nidulans tpiA gene (McKnight, G. L. et al. (1986) Cell 46, 143-147), the A. nidulans amdS gene (Hynes, M. J. et al. (1983) Mol. Cell Biol. 3, 1430-1439), the H. jecorina xln1 gene, the H. jecorina cbh2 gene, the H. jecorina eg1 gene, the H. jecorina eg2 gene, the H. jecorina eg3 gene, and higher eukaryotic promoters such as the SV40 early promoter (Barclay, S. L. and E. Meller (1983) Molecular and Cellular Biology 3, 2117-2130).

A nucleic acid is “operably linked” when it is placed into a functional relationship with another nucleic acid sequence. For example, DNA encoding a secretory leader, i.e., a signal peptide, is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. Thus, the term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “signal sequence”, “signal peptide”, “secretory sequence”, “secretory peptide”, “secretory signal sequence”, “secretory signal peptide” and the like denotes a peptide sequence that, as a component of a larger polypeptide, directs the larger polypeptide through a secretory pathway of a cell in which it is synthesized, as well as nucleic acids encoding such peptides. In general, the larger polypeptide (or protein) is commonly cleaved to remove the secretory/signal peptide during transit through the secretory pathway, where the cleaved form of the polypeptide (i.e., the form without the signal/secretory peptide) is often referred to herein as the “mature form” of the polypeptide. For example, SEQ ID NO:2 provides the amino acid sequence of CBH1 from H. jecorina with the signal peptide while SEQ ID NO:3 provides the amino acid sequence of the mature form of CBH1 from H. jecorina, i.e., without the signal peptide.

As used herein, the term “vector” refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art.

Accordingly, an “expression cassette” or “expression vector” is a nucleic acid construct generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter.

As used herein, the term “plasmid” refers to a circular double-stranded (ds) DNA construct that forms an extrachromosomal self-replicating genetic element when present in many bacteria and some eukaryotes. Plasmids may be employed for any of a number of different purposes, e.g., as cloning vectors, propagation vectors, expression vectors, etc.

As used herein, the term “selectable marker” refers to a nucleotide sequence or polypeptide encoded thereby which is capable of expression in cells and where expression of the selectable marker in cells confers the ability to be differentiated from cells that do not express the selectable marker. In certain embodiments, a selectable marker allows a cell expressing it to grow in the presence of a corresponding selective agent, or under corresponding selective growth conditions. In other embodiments, a selectable marker allows a cell expressing it to be identified and/or isolated from cells that do not express it by virtue of a physical characteristic, e.g., by differences in fluorescence, immuno-reactivity, etc.

In general, nucleic acid molecules which encode the variant CBH1 will hybridize, under moderate to high stringency conditions to the wild type sequence provided herein as SEQ ID NO:1 (native H. jecorina CBH1). However, in some cases a CBH1-encoding nucleotide sequence is employed that possesses a substantially different codon usage, while the enzyme encoded by the CBH1-encoding nucleotide sequence has the same or substantially the same amino acid sequence as the native enzyme. For example, the coding sequence may be modified to facilitate faster expression of CBH1 in a particular prokaryotic or eukaryotic expression system, in accordance with the frequency with which a particular codon is utilized by the host (commonly referred to as “codon optimization”). Te'o, et al. (2000), for example, describes the optimization of genes for expression in filamentous fungi. Such nucleic acid sequences are sometimes referred to as “degenerate” or “degenerated sequences”.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (5° below the Tm of the probe); “high stringency” at about 5-10° below the Tm; “moderate” or “intermediate stringency” at about 10-20° below the Tm of the probe; and “low stringency” at about 20-25° below the Tm. Functionally, maximum stringency conditions may be used to identify sequences having strict identity or near-strict identity with the hybridization probe; while high stringency conditions are used to identify sequences having about 80% or more sequence identity with the probe.

Moderate and high stringency hybridization conditions are well known in the art (see, for example, Sambrook, et al, 1989, Chapters 9 and 11, and in Ausubel, F. M., et al., 1993, expressly incorporated by reference herein). An example of high stringency conditions includes hybridization at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C.

As used herein, the terms “transformed”, “stably transformed” or “transgenic” with reference to a cell means the cell has a non-native (heterologous) nucleic acid sequence integrated into its genome or as an episomal plasmid that is maintained through multiple generations.

As used herein, the term “expression” refers to the process by which a polypeptide is produced based on the nucleic acid sequence of a gene. The process generally includes both transcription and translation.

The term “introduced” in the context of inserting a nucleic acid sequence into a cell, means “transfection”, or “transformation” or “transduction” and includes reference to the incorporation of a nucleic acid sequence into a eukaryotic or prokaryotic cell where the nucleic acid sequence may be incorporated into the genome of the cell (for example, chromosome, plasmid, plastid, or mitochondrial DNA), converted into an autonomous replicon, or transiently expressed (for example, transfected mRNA).

It follows that the term “desired cellulase expression” refers to transcription and translation of the desired cellulase gene, the products of which include precursor RNA, mRNA, polypeptide, post-translationally processed polypeptides. By way of example, assays for CBH1 expression include Western blot for CBH1 enzyme, Northern blot analysis and reverse transcriptase polymerase chain reaction (RT-PCR) assays for CBH1 mRNA, and endoglucanase activity assays as described in Shoemaker S. P. and Brown R. D. Jr. (Biochim. Biophys. Acta, 1978, 523:133-146) and Schulein (1988).

By the term “host cell” is meant a cell that contains a vector and supports the replication, and/or transcription and/or transcription and translation (expression) of the expression construct. Host cells for use in the present invention can be prokaryotic cells, such as E. coli, or eukaryotic cells such as yeast, plant, insect, amphibian, or mammalian cells. In certain embodiments, host cells are filamentous fungi.

As used herein, the term “detergent composition” refers to a mixture which is intended for use in a wash medium for the laundering of soiled cellulose containing fabrics. In the context of the present invention, such compositions may include, in addition to cellulases and surfactants, additional hydrolytic enzymes, builders, bleaching agents, bleach activators, bluing agents and fluorescent dyes, caking inhibitors, masking agents, cellulase activators, antioxidants, and solubilizers.

As used herein, the term “surfactant” refers to any compound generally recognized in the art as having surface active qualities. Thus, for example, surfactants comprise anionic, cationic and nonionic surfactants such as those commonly found in detergents. Anionic surfactants include linear or branched alkylbenzenesulfonates; alkyl or alkenyl ether sulfates having linear or branched alkyl groups or alkenyl groups; alkyl or alkenyl sulfates; olefinsulfonates; and alkanesulfonates. Ampholytic surfactants include quaternary ammonium salt sulfonates, and betaine-type ampholytic surfactants. Such ampholytic surfactants have both the positive and negative charged groups in the same molecule. Nonionic surfactants may comprise polyoxyalkylene ethers, as well as higher fatty acid alkanolamides or alkylene oxide adduct thereof, fatty acid glycerine monoesters, and the like.

As used herein, the term “cellulose containing fabric” refers to any sewn or unsewn fabrics, yarns or fibers made of cotton or non-cotton containing cellulose or cotton or non-cotton containing cellulose blends including natural cellulosics and manmade cellulosics (such as jute, flax, ramie, rayon, and lyocell).

As used herein, the term “cotton-containing fabric” refers to sewn or unsewn fabrics, yarns or fibers made of pure cotton or cotton blends including cotton woven fabrics, cotton knits, cotton denims, cotton yarns, raw cotton and the like.

As used herein, the term “stonewashing composition” refers to a formulation for use in stonewashing cellulose containing fabrics. Stonewashing compositions are used to modify cellulose containing fabrics prior to sale, i.e., during the manufacturing process. In contrast, detergent compositions are intended for the cleaning of soiled garments and are not used during the manufacturing process.

When an amino acid position (or residue) in a first polypeptide is noted as being “equivalent” to an amino acid position in a second, related polypeptide, it means that the amino acid position of the first polypeptide corresponds to the position noted in the second, related polypeptide by one or more of (i) primary sequence alignment (see description of sequence alignment and sequence identity below); (ii) structural sequence homology; or (iii) analogous functional property. Thus, an amino acid position in a first CBH enzyme (or a variant thereof) can be identified as “equivalent” (or “homologous”) to an amino acid position in a second CBH enzyme (or even multiple different CBH enzymes).

Primary Sequence Alignment:

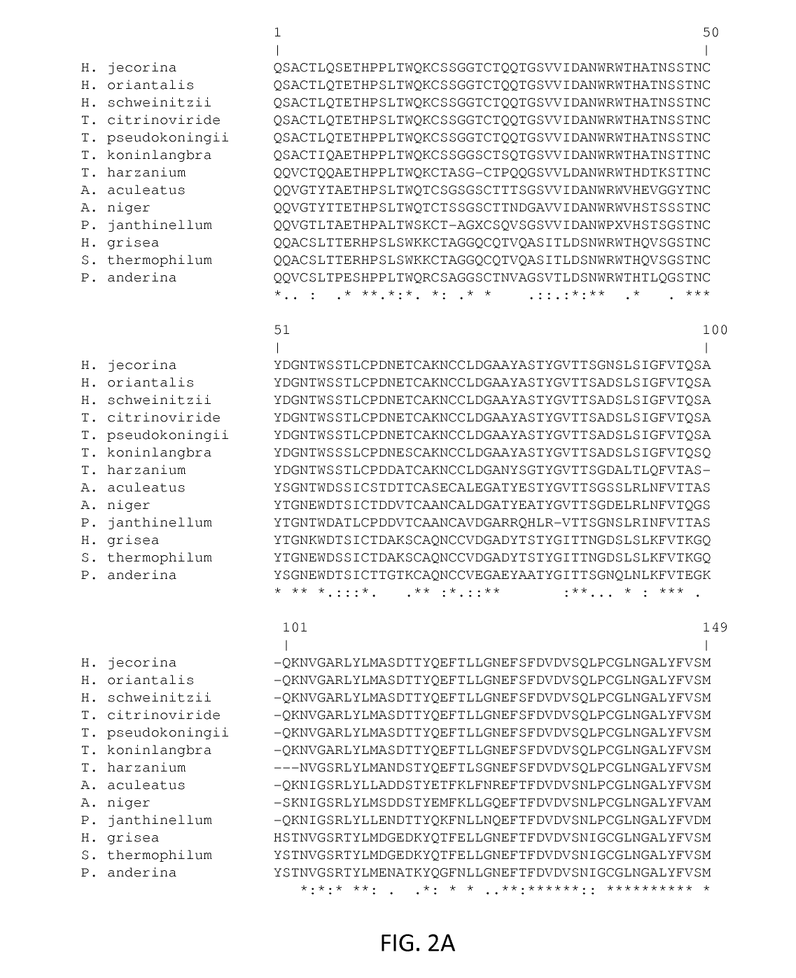

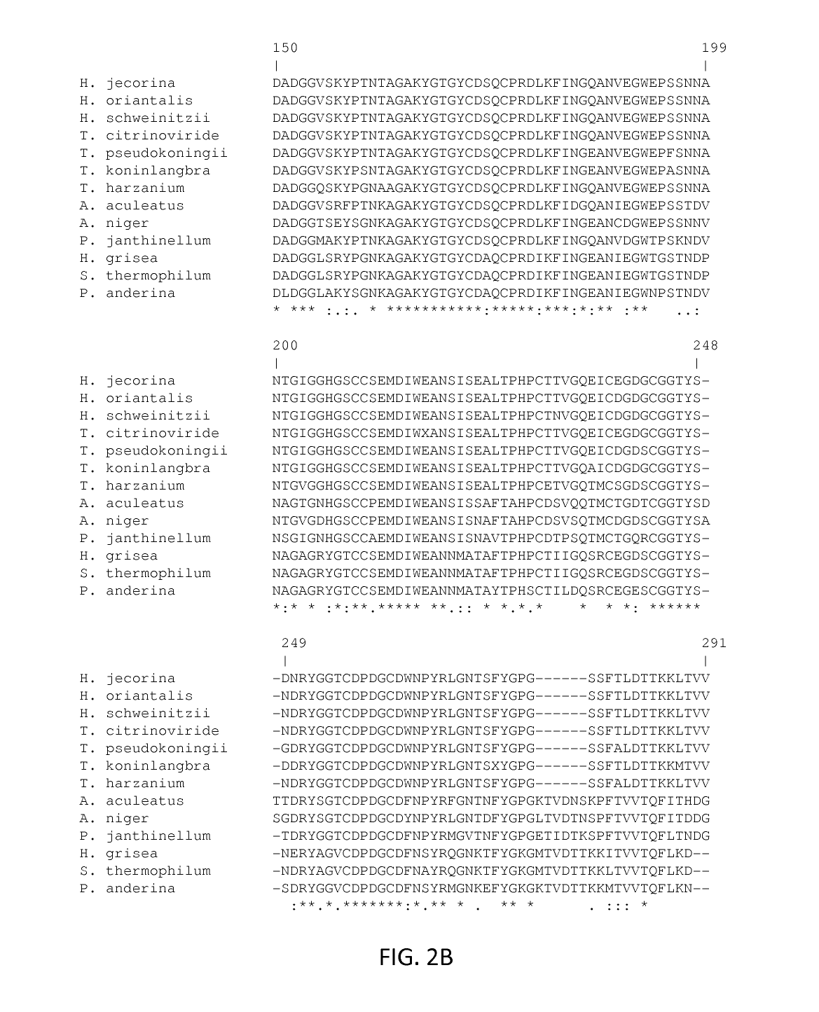

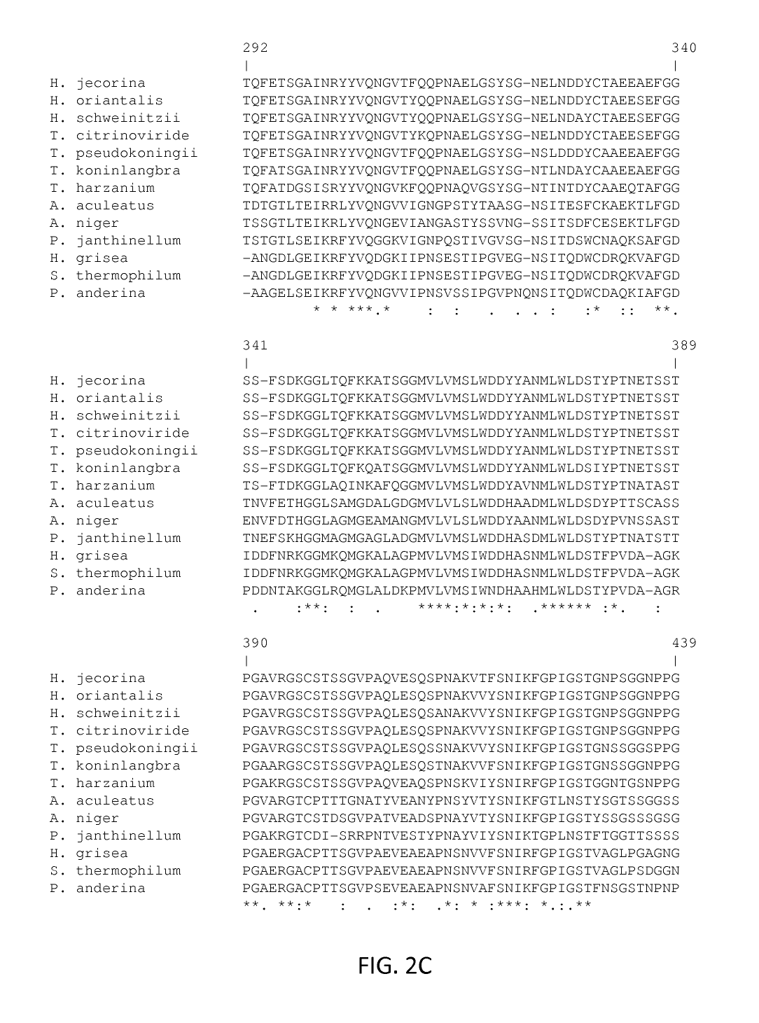

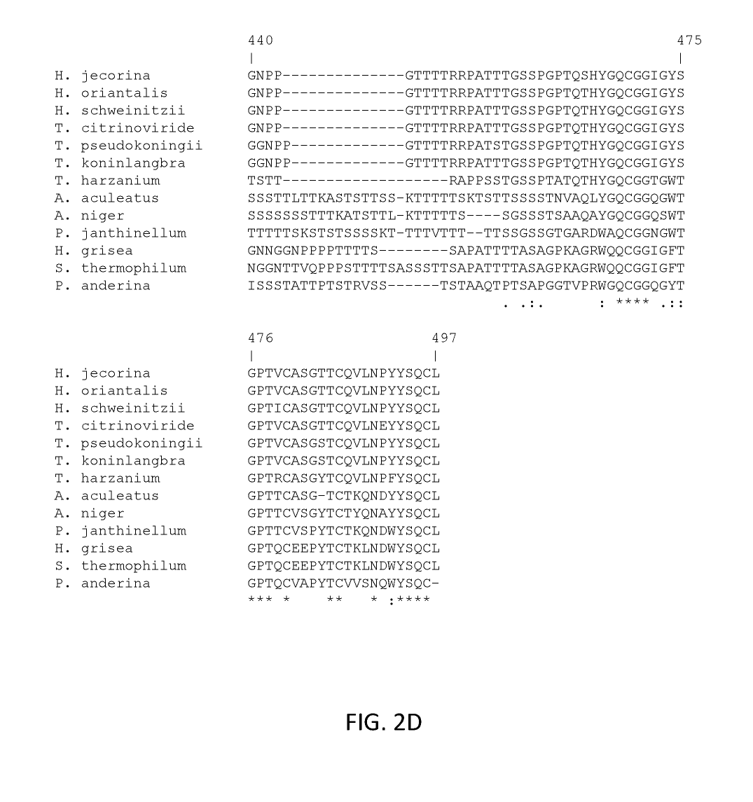

Equivalent amino acid positions can be determined using primary amino acid sequence alignment methodologies, many of which are known in the art. For example, by aligning the primary amino acid sequences of two or more different CBH enzymes, it is possible to designate an amino acid position number from one CBH enzyme as equivalent to the position number of another one of the aligned CBH enzymes. In this manner, the numbering system originating from the amino acid sequence of one CBH enzyme (e.g., the CBH1 enzyme denoted in SEQ ID NO: 3) can be used to identify equivalent (or homologous) amino acid residues in other CBH enzymes (e.g., the CBH1 enzymes denoted in SEQ ID NOs: 4 to 15; see FIG. 2).

Structural Sequence Homology:

In addition to determining “equivalent” amino acid positions using primary sequence alignment methodologies, “equivalent” amino acid positions may also be defined by determining homology at the level of secondary and/or tertiary structure. For example, for a cellulase whose tertiary structure has been determined by x-ray crystallography, equivalent residues can be defined as those for which the atomic coordinates of two or more of the main chain atoms of a particular amino acid residue of the cellulase are within 0.13 nm and preferably 0.1 nm after alignment with Hypocrea jecorina CBH1 (N on N, CA on CA, C on C, and 0 on 0). Alignment is achieved after the best model has been oriented and positioned to give the maximum overlap of atomic coordinates of non-hydrogen protein atoms of the cellulase in question to the H. jecorina CBH1. The best model is the crystallographic model giving the lowest R factor for experimental diffraction data at the highest resolution available.

$$R factor = \frac{{Σ}_{h} \vert Fo ( h ) \vert - \vert Fc ( h ) \vert}{{Σ}_{h} \vert Fo ( h ) \vert}$$

Analogous Functional Property:

Equivalent amino acid residues in a first polypeptide which are functionally analogous to a specific residue of a second related polypeptide (e.g., a first cellulase and H. jecorina CBH1) are defined as those amino acids in the first polypeptide that adopt a conformation such that they alter, modify, or contribute to polypeptide structure, substrate binding, or catalysis in a manner defined and attributed to a specific residue of the second related polypeptide (e.g., H. jecorina CBH1). When a tertiary structure has been obtained by x-ray crystallography for the first polypeptide, amino acid residues of the first polypeptide that are functionally analogous to the second polypeptide occupy an analogous position to the extent that, although the main chain atoms of the given residue may not satisfy the criteria of equivalence on the basis of occupying a homologous position, the atomic coordinates of at least two of the side chain atoms of the residue lie with 0.13 nm of the corresponding side chain atoms of the second polypeptide (e.g., H. jecorina CBH1).

The term “improved property” or “improved performance” and the like with respect to a variant enzyme (e.g., a CBH variant) is defined herein as a characteristic or activity associated with a variant enzyme which is improved as compared to its respective parent enzyme. Improved properties include, but are not limited to, improved thermostability or altered temperature-dependent activity profile, improved activity or stability at a desired pH or pH range, improved substrate specificity, improved product specificity, and improved stability in the presence of a chemical or other component in a cellulase process step, etc. Improved performance may be determined using a particular assay(s) including, but not limited to: (a) Expression (Protein Content Determination assay), (b) PASC Hydrolysis Assay, (c) PASC Hydrolysis Assay in the Presence of EG2, (d) PASC Hydrolysis Assay After Heat Incubation, (e) Whole Hydrolysate PCS (whPCS) Assay, (f) Dilute Ammonia Corn Cob (daCC) Assay, and (g) Dilute Ammonia Corn Stover (daCS) assay.

The term “improved thermostability” with respect to a variant protein (e.g., a CBH variant) is defined herein as a variant enzyme displaying retention of enzymatic activity after a period of incubation at an elevated temperature relative to the parent enzyme. Such a variant may or may not display an altered thermal activity profile relative to the parent. For example, a variant may have an improved ability to refold following incubation at elevated temperature relative to the parent.

By “improved product specificity” is meant a variant enzyme displaying an altered product profile as compared to the parent enzyme, where the altered product profile of the variant is improved in a given application as compared to the parent. A “product profile” is defined herein as the chemical composition of the reaction products produced by the enzyme of interest.

By “improved chemical stability” is meant that a variant enzyme displays retention of enzymatic activity after a period of incubation in the presence of a chemical or chemicals that reduce the enzymatic activity of the parent enzyme under the same conditions. Variants with improved chemical stability are better able to catalyze a reaction in the presence of such chemicals as compared to the parent enzyme.

A “pH range,” with reference to an enzyme, refers to the range of pH values under which the enzyme exhibits catalytic activity.

The terms “pH stable” and “pH stability,” with reference to an enzyme, relate to the ability of the enzyme to retain activity over a wide range of pH values for a predetermined period of time (e.g., 15 min., 30 min., 1 hour).

“Percent sequence identity” or grammatical equivalents means that a particular sequence has at least a certain percentage of amino acid residues identical to those in a specified reference sequence using an alignment algorithm. An example of an algorithm that is suitable for determining sequence similarity is the BLAST algorithm, which is described in Altschul, et al., J. Mol. Biol. 215:403-410 (1990). Software for performing BLAST analyses is publicly available through the National Center for Biotechnology Information (<www(dot)ncbi(dot)nlm(dot)nih(dot)gov>). This algorithm involves first identifying high scoring sequence pairs (HSPs) by identifying short words of length W in the query sequence that either match or satisfy some positive-valued threshold score T when aligned with a word of the same length in a database sequence. These initial neighborhood word hits act as starting points to find longer HSPs containing them. The word hits are expanded in both directions along each of the two sequences being compared for as far as the cumulative alignment score can be increased. Extension of the word hits is stopped when: the cumulative alignment score falls off by the quantity X from a maximum achieved value; the cumulative score goes to zero or below; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff & Henikoff, Proc. Natl. Acad. Sci. USA 89:10915 (1989)) alignments (B) of 50, expectation (E) of 10, M′5, N′-4, and a comparison of both strands.

The BLAST algorithm then performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a protease if the smallest sum probability in a comparison of the test amino acid sequence to a protease amino acid sequence is less than about 0.1, more preferably less than about 0.01, and most preferably less than about 0.001.

When questions of percent sequence identity arise, alignment using the CLUSTAL W algorithm with default parameters will govern. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF.

### II. Molecular Biology

Embodiments of the subject invention provide for the expression of a desired cellulase enzyme (or combination of cellulase enzymes) from cellulase-encoding nucleic acids under control of a promoter functional in a host cell of interest, e.g., a filamentous fungus. Therefore, this invention relies on a number of routine techniques in the field of recombinant genetics. Basic texts disclosing examples of suitable recombinant genetics methods are noted above.

Any method known in the art that can introduce mutations into a parent nucleic acid/polypeptide is contemplated by the present invention.

The present invention relates to the expression, purification and/or isolation and use of variant CBH1 enzymes. These enzymes may be prepared by recombinant methods utilizing any of a number of cbh1 genes known in the art (e.g., the cbh1 gene in SEQ ID NOs:3 to 15, e.g., from H. jecorina). Any convenient method for introducing mutations may be employed, including site directed mutagenesis. As indicated above, mutations (or variations) include substitutions, additions, deletions or truncations that will correspond to one or more amino acid change in the expressed CBH1 variant. Again, site directed mutagenesis and other methods of incorporating amino acid changes in expressed polypeptides at the DNA level can be found in numerous references, e.g., Green and Sambrook, et al. 2012 and Ausubel, et al.

DNA encoding an amino acid sequence variant of a parent CBH1 is prepared by a variety of methods known in the art. These methods include, but are not limited to, preparation by site-directed (or oligonucleotide-mediated) mutagenesis, PCR mutagenesis, and cassette mutagenesis of an earlier prepared DNA encoding the parent CBH1 enzyme.

Site-directed mutagenesis is one method that can be employed in preparing substitution variants. This technique is well known in the art (see, e.g., Carter et al. Nucleic Acids Res. 13:4431-4443 (1985) and Kunkel et al., Proc. Natl. Acad. Sci. USA 82:488 (1987)). Briefly, in carrying out site-directed mutagenesis of DNA, the starting DNA is altered by first hybridizing an oligonucleotide encoding the desired mutation to a single strand of such starting DNA. After hybridization, a DNA polymerase is used to synthesize an entire second strand, using the hybridized oligonucleotide as a primer, and using the single strand of the starting DNA as a template. Thus, the oligonucleotide encoding the desired mutation is incorporated in the resulting double-stranded DNA.

PCR mutagenesis is also suitable for making amino acid sequence variants of the parent CBH1. See Higuchi, in PCR Protocols, pp. 177-183 (Academic Press, 1990); and Vallette et al., Nuc. Acids Res. 17:723-733 (1989). Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template.

Another method for preparing variants, cassette mutagenesis, is based on the technique described by Wells et al., Gene 34:315-323 (1985). The starting material is the plasmid (or other vector) comprising the starting polypeptide DNA to be mutated. The codon(s) in the starting DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the starting polypeptide DNA. The plasmid DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated DNA sequence.

Alternatively, or additionally, the desired amino acid sequence encoding a desired cellulase can be determined, and a nucleic acid sequence encoding such amino acid sequence variant can be generated synthetically.

The desired cellulase(s) so prepared may be subjected to further modifications, oftentimes depending on the intended use of the cellulase. Such modifications may involve further alteration of the amino acid sequence, fusion to heterologous polypeptide(s) and/or covalent modifications.

### III. Variant CBH1 Polypeptides and Nucleic Acids Encoding Same

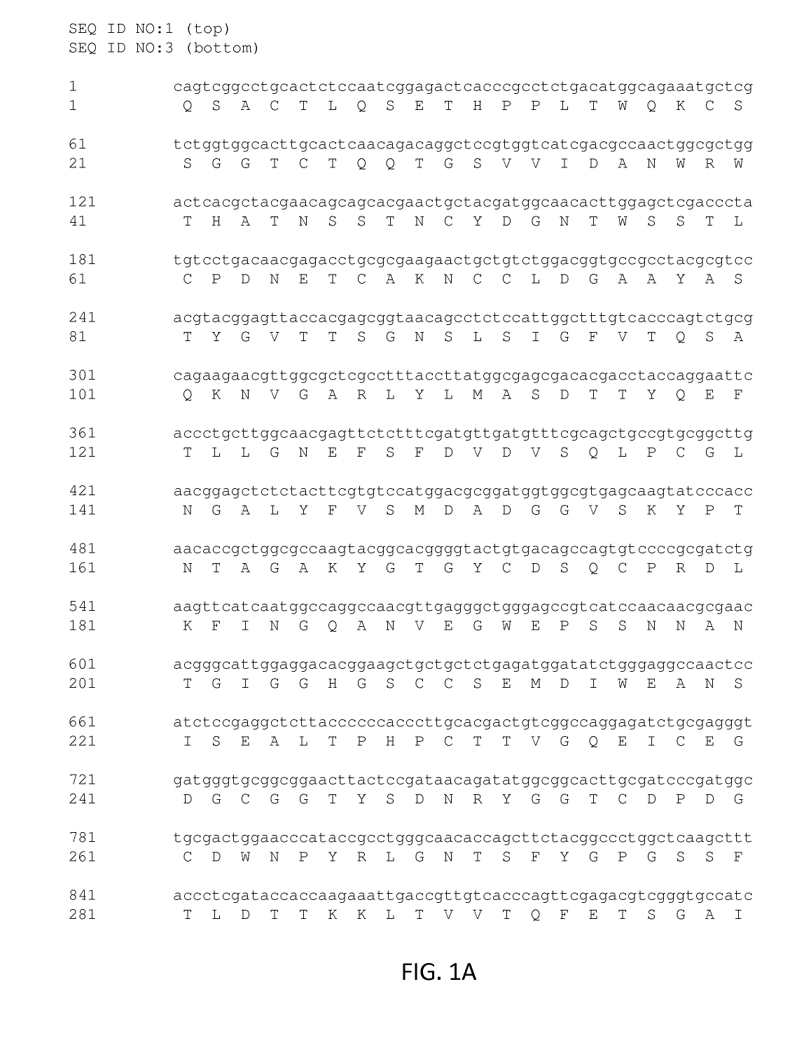

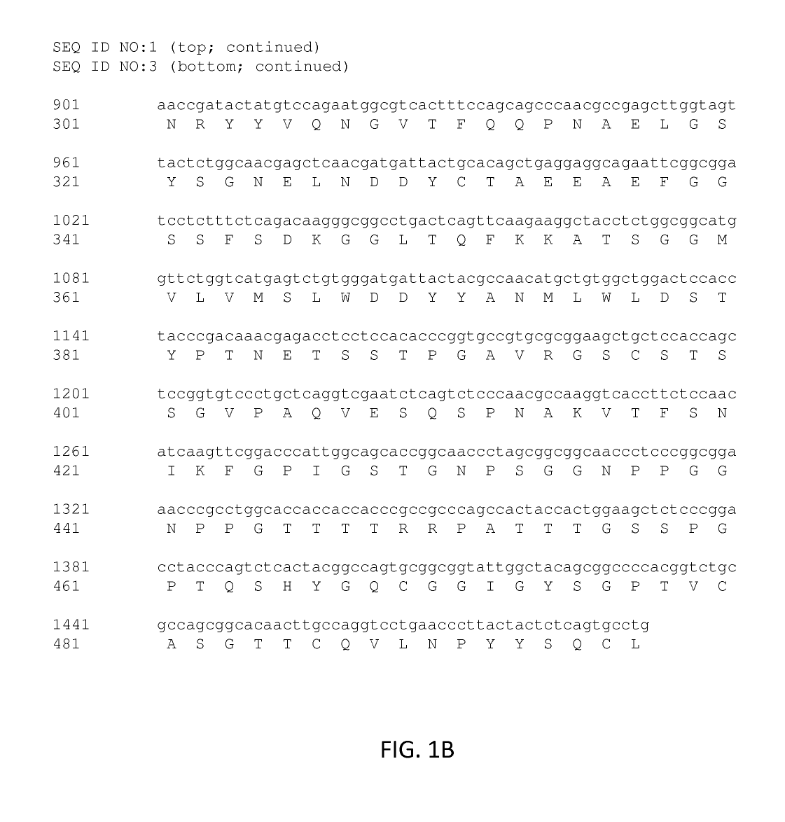

In one aspect, variant CBH enzymes are provided. The variant CBH enzymes have one or more mutations, as set forth herein, with respect to a parent CBH enzyme that has at least 80% (i.e., 80% or greater) amino acid sequence identity to H. jecorina CBH1 (SEQ ID NO: 3), including at least 81%, 82%, 83%, 84%, 85%, 86%, 87%, 88%, 89%, 90%, 91%, 92%, 93%, 94%, 95%, 96%, 97%, 98%, 99%, up to and including 100% amino acid sequence identity to SEQ ID NO:3. In certain embodiments, the parent CBH is a fungal cellobiohydrolase 1 (CBH1), for example fungal CBH1 enzymes from Hypocrea jecorina, Hypocrea schweinitzii, Hypocrea orientalis, Trichoderma pseudokoningii, Trichoderma konilangbra, Trichoderma citrinoviride, Trichoderma harzanium, Aspergillus aculeatus, Aspergillus niger; Penicillium janthinellum, Humicola grisea, Scytalidium thermophilum, or Podospora anderina. Further, the variant CBH enzyme has cellulase activity, where in certain embodiments, the variant CBH has an improved property as compared to the parent CBH (as detailed herein). The amino acid sequence for the wild type, mature form of H. jecorina CBH1 is shown in FIG. 1.

In certain embodiments, a variant CBH enzyme comprises an amino acid mutation at one or more amino acid positions corresponding to residues F418, T246, T255, D241, G234, P194, N200, N49, Y247, T356, S92, and T41 in the mature form of CBH1 from H. jecorina (SEQ ID NO:3). Because certain parent CBH enzymes according to aspects of the invention may not have the same amino acid as wild type CBH1 from H. jecorina, amino acid positions corresponding to the residues noted above may also be designated either by the position number alone (i.e., 418, 246, 255, 241, 234, 194, 200, 49, 247, 356, 92, and 41) or with an “X” prefix (i.e., X418, X246, X255, X241, X234, X194, X200, X49, X247, X356, X92, and X41). It is noted here that all three ways of designating the amino acid positions corresponding to a specific amino acid residue in CBH1 from H. jecorina are interchangeable.

The amino acid sequence of the CBH variant differs from the parent CBH amino acid sequence by the substitution, deletion or insertion of one or more amino acids of the parent amino acid sequence. A residue (amino acid) of a CBH variant is equivalent to a residue of Hypocrea jecorina CBH1 if it is either homologous (i.e., corresponding in position in either primary or tertiary structure) or is functionally analogous to a specific residue or portion of that residue in Hypocrea jecorina CBH1 (i.e., having the same or similar functional capacity to combine, react, or interact chemically or structurally). As used herein, numbering is intended to correspond to that of the mature CBH1 amino acid sequence as illustrated in FIG. 1.

Alignment of amino acid sequences to determine homology can be determined by using a “sequence comparison algorithm.” Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), by visual inspection or MOE by Chemical Computing Group, Montreal Canada. See also the description of “percent sequence identity” provided in the Definitions section above.

In certain embodiments, the mutation(s) in a variant CBH enzyme is an amino acid substitution at one or more site corresponding to amino acid position F418, T246, T255, D241, G234, P194, N200, N49, Y247, T356, S92, and T41 in CBH1 from H. jecorina (SEQ ID NO:3), where in some embodiments, the substitutions are selected from the following group: F418M, T246S, T255V, D241N, G234D, P194V, T255I, T255K, T255R, N200G, N49P, T246V, Y247D, N200R, T246P, T255D, T356L, S92T, T255P, T41I. All possible combinations of the aforementioned substitutions at the indicated sites (i.e., having 1, 2, 3, 4, 5, 6, 7, 8, 9, 10, 11 or 12 substitutions) are contemplated embodiments of the invention, including but not limited to the following:
1. CBH variant having any single amino acid substitution selected from: F418M, T246S, T255V, D241N, G234D, P194V, T255I, T255K, T255R, N200G, N49P, T246V, Y247D, N200R, T246P, T255D, T356L;
2. CBH variant of 1 above having a F418M substitution;
3. CBH variant of 1 or 2 above having a T246S substitution;
4. CBH variant of 1 or 2 above having a T246P substitution.
5. CBH variant of 1 or 2 above having a T246V substitution;
6. CBH variant of 1, 2, 3, 4 or 5 above having a T255V substitution;
7. CBH variant of 1, 2, 3, 4 or 5 above having a T255I substitution;
8. CBH variant of 1, 2, 3, 4 or 5 above having a T255K substitution;
9. CBH variant of 1, 2, 3, 4 or 5 above having a T255R substitution;
10. CBH variant of 1, 2, 3, 4 or 5 above having a T255D substitution;
11. CBH variant of 1, 2, 3, 4 or 5 above and further including a T255P substitution;
12. CBH variant of 1, 2, 3, 4, 5, 6, 7, 8, 9, 10 or 11 above having a D241N substitution;
13. CBH variant of 1, 2, 3, 4, 5, 6, 7, 8, 9, 10, 11 or 12 above having a G234D substitution;
14. CBH variant of 1, 2, 3, 4, 5, 6, 7, 8, 9, 10, 11, 12 or 13 above having a P194V substitution;
15. CBH variant of 1, 2, 3, 4, 5, 6, 7, 8, 9, 10, 11, 12, 13 or 14 above having a N200G substitution;
16. CBH variant of 1, 2, 3, 4, 5, 6, 7, 8, 9, 10, 11, 12, 13 or 14 above having a N200R substitution;
17. CBH variant of 1, 2, 3, 4, 5, 6, 7, 8, 9, 10, 11, 12, 13, 14, 15 or 16 above having a N49P substitution;
18. CBH variant of 1, 2, 3, 4, 5, 6, 7, 8, 9, 10, 11, 12, 13, 14, 15, 16 or 17 above having a Y247D substitution; and
19. CBH variant of 1, 2, 3, 4, 5, 6, 7, 8, 9, 10, 11, 12, 13, 14, 15, 16, 17 or 18 above having a T356L substitution.

In certain embodiments, a variant CBH enzyme as described above further includes an additional amino acid mutation at one or both amino acid positions corresponding to S92 and T41 of SEQ ID NO:3, where in certain of these embodiments the mutation(s) is a substitution selected from: S92T and T41I.

All possible combinations of these additional mutations with the substitutions described above are contemplated embodiments of the invention, including but not limited to the following:
20. CBH variant of 1, 2, 3, 4, 5, 6, 7, 8, 9, 10, 11, 12, 13, 14, 15, 16, 17 or 18 above and further including a S92T substitution;
21. CBH variant of 1, 2, 3, 4, 5, 6, 7, 8, 9, 10, 11, 12, 13, 14, 15, 16, 17, 18 or 19 above and further including a T41I substitution.

In a further embodiment, a CBH1 variant as described above includes an additional

In another aspect, nucleic acids encoding a variant CBH enzyme having one or more mutations with respect to a parent CBH enzyme (e.g., as described above) are provided. In certain embodiments, the parent CBH1 has at least 80% (i.e., 80% or greater) amino acid sequence identity to H. jecorina CBH1 (SEQ ID NO:3). In certain embodiments, the nucleic acid encoding a variant CBH enzyme is at least 40%, at least 50%, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or even at least 99% homology/identity to SEQ ID NO: 1 (excluding the portion of the nucleic acid that encodes the signal sequence). It will be appreciated that due to the degeneracy of the genetic code, a plurality of nucleic acids may encode the same variant CBH enzyme. Moreover, nucleic acids encoding a variant CBH enzyme as described herein may be engineered to be codon optimized, e.g., to improve expression in a host cell of interest. Certain codon optimization techniques are known in the art.

In certain embodiments, the variant CBH enzyme-encoding nucleic acid hybridizes under stringent conditions to a nucleic acid encoding (or complementary to a nucleic acid encoding) a CBH having at least 40%, at least 50%, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or even at least 99% homology/identity to SEQ ID NO: 1 (excluding the portion of the nucleic acid that encodes the signal sequence).

Nucleic acids may encode a “full-length” (“fl” or “FL”) variant CBH enzyme, which includes a signal sequence, only the mature form of a variant CBH enzyme, which lacks the signal sequence, or a truncated form of a variant CBH enzyme, which lacks portions of the N and/or C-terminus of the mature form.

A nucleic acid that encodes a variant CBH enzyme can be operably linked to various promoters and regulators in a vector suitable for expressing the variant CBH enzyme in a host cell(s) of interest, as described below.

### IV. Expression of Recombinant CBH1 Variants

Aspects of the subject invention include methods and compositions related to the generation nucleic acids encoding CBH variants, host cells containing such nucleic acids, the production of CBH variants by such host cells, and the isolation, purification and/or use of the CBH variants.

As such, embodiments of the invention provide host cells that have been transduced, transformed or transfected with an expression vector comprising a desired CBH variant-encoding nucleic acid sequence. For example, a filamentous fungal cell or yeast cell is transfected with an expression vector having a promoter or biologically active promoter fragment or one or more (e.g., a series) of enhancers which functions in the host cell line, operably linked to a DNA segment encoding a desired CBH variant, such that desired CBH variant is expressed in the cell line.

A. Nucleic Acid Constructs/Expression Vectors.

Natural or synthetic polynucleotide fragments encoding a desired CBH variant may be incorporated into heterologous nucleic acid constructs or vectors, capable of introduction into, and replication in, a host cell of interest (e.g., a filamentous fungal or yeast cell). The vectors and methods disclosed herein are suitable for use in host cells for the expression of a desired CBH variant. Any vector may be used as long as it meets the desired replication/expression characteristics in the host cell(s) into which it is introduced (such characteristics generally being defined by the user). Large numbers of suitable vectors and promoters are known to those of skill in the art, some of which are commercially available. Cloning and expression vectors are also described in Sambrook et al., 1989, Ausubel F M et al., 1989, and Strathern et al., 1981, each of which is expressly incorporated by reference herein. Appropriate expression vectors for fungi are described in van den Hondel, C. A. M. J. J. et al. (1991) In: Bennett, J. W. and Lasure, L. L. (eds.) More Gene Manipulations in Fungi. Academic Press, pp. 396-428. The appropriate DNA sequence may be inserted into a plasmid or vector (collectively referred to herein as “vectors”) by a variety of procedures. In general, the DNA sequence is inserted into an appropriate restriction endonuclease site(s) by standard procedures. Such procedures and related sub-cloning procedures are deemed to be within the scope of knowledge of those skilled in the art.

Recombinant host cells comprising the coding sequence for a desired CBH variant may be produced by introducing a heterologous nucleic acid construct comprising the desired CBH variant coding sequence into the desired host cells (e.g., as described in further detail below). For example, a desired CBH variant coding sequence may be inserted into a suitable vector according to well-known recombinant techniques and used to transform a filamentous fungi capable of CBH expression. As has been noted above, due to the inherent degeneracy of the genetic code, other nucleic acid sequences which encode substantially the same or a functionally equivalent amino acid sequence may be used to clone and express a desired CBH variant. Therefore it is appreciated that such substitutions in the coding region fall within the sequence variants covered by the present invention.

The present invention also includes recombinant nucleic acid constructs comprising one or more of the desired CBH variant-encoding nucleic acid sequences as described above. The constructs comprise a vector, such as a plasmid or viral vector, into which a sequence of the invention has been inserted, in a forward or reverse orientation.

Heterologous nucleic acid constructs may include the coding sequence for a desired CBH variant: (i) in isolation; (ii) in combination with additional coding sequences; such as fusion polypeptide or signal peptide coding sequences, where the desired CBH variant coding sequence is the dominant coding sequence; (iii) in combination with non-coding sequences, such as introns and control elements, such as promoter and terminator elements or 5′ and/or 3′ untranslated regions, effective for expression of the coding sequence in a suitable host; and/or (iv) in a vector or host environment in which the desired CBH variant coding sequence is a heterologous gene.

In one aspect of the present invention, a heterologous nucleic acid construct is employed to transfer a desired CBH variant-encoding nucleic acid sequence into a host cell in vitro, e.g., into established filamentous fungal and yeast lines. Long-term production of a desired CBH variant can be achieved by generating a host cell that has stable expression of the CBH variant. Thus, it follows that any method effective to generate stable transformants may be used in practicing the invention.

Appropriate vectors are typically equipped with a selectable marker-encoding nucleic acid sequence, insertion sites, and suitable control elements, such as promoter and termination sequences. The vector may comprise regulatory sequences, including, for example, non-coding sequences, such as introns and control elements, i.e., promoter and terminator elements or 5′ and/or 3′ untranslated regions, effective for expression of the coding sequence in host cells (and/or in a vector or host cell environment in which a modified soluble protein antigen coding sequence is not normally expressed), operably linked to the coding sequence. Large numbers of suitable vectors and promoters are known to those of skill in the art, many of which are commercially available and/or are described in Sambrook, et al., (supra).

Examples of suitable promoters include both constitutive promoters and inducible promoters, examples of which include a CMV promoter, an SV40 early promoter, an RSV promoter, an EF-1 a promoter, a promoter containing the tet responsive element (TRE) in the tet-on or tet-off system as described (ClonTech and BASF), the beta actin promoter and the metallothionine promoter that can upregulated by addition of certain metal salts. A promoter sequence is a DNA sequence which is recognized by the particular host cell for expression purposes. It is operably linked to DNA sequence encoding a variant CBH1 polypeptide. Such linkage comprises positioning of the promoter with respect to the initiation codon of the DNA sequence encoding the variant CBH1 polypeptide in the expression vector such that the promoter can drive transcription/translation of the CBH variant-encoding sequence. The promoter sequence contains transcription and translation control sequence which mediate the expression of the variant CBH1 polypeptide. Examples include the promoters from the Aspergillus niger, A awamori or A. oryzae glucoamylase, alpha-amylase, or alpha-glucosidase encoding genes; the A. nidulans gpdA or trpC Genes; the Neurospora crassa cbh1 or trp1 genes; the A. niger or Rhizomucor miehei aspartic proteinase encoding genes; the H. jecorina cbh1, cbh2, egl1, egl2, or other cellulase encoding genes.

The choice of the proper selectable marker will depend on the host cell, and appropriate markers for different hosts are well known in the art. Typical selectable marker genes include argB from A. nidulans or H. jecorina, amdS from A. nidulans, pyr4 from Neurospora crassa or H. jecorina, pyrG from Aspergillus niger or A. nidulans. Additional examples of suitable selectable markers include, but are not limited to trpc, trp1, oliC31, niaD or leu2, which are included in heterologous nucleic acid constructs used to transform a mutant strain such as trp-, pyr-, leu- and the like.

Such selectable markers confer to transformants the ability to utilize a metabolite that is usually not metabolized by the filamentous fungi. For example, the amdS gene from H. jecorina which encodes the enzyme acetamidase that allows transformant cells to grow on acetamide as a nitrogen source. The selectable marker (e.g. pyrG) may restore the ability of an auxotrophic mutant strain to grow on a selective minimal medium or the selectable marker (e.g. olic31) may confer to transformants the ability to grow in the presence of an inhibitory drug or antibiotic.

The selectable marker coding sequence is cloned into any suitable plasmid using methods generally employed in the art. Examples of suitable plasmids include pUC18, pBR322, pRAX and pUC100. The pRAX plasmid contains AMA1 sequences from A. nidulans, which make it possible to replicate in A. niger.

The practice of the present invention will employ, unless otherwise indicated, conventional techniques of molecular biology, microbiology, recombinant DNA, and immunology, which are within the skill of the art. Such techniques are explained fully in the literature. See, for example, Sambrook et al., 1989; Freshney, 1987; Ausubel, et al., 1993; and Coligan et al., 1991.

B. Host Cells and Culture Conditions for CBH1 and Variant CBH1 Enzyme Production

After DNA sequences that encode the CBH1 variants have been cloned into DNA constructs, the DNA is used to transform microorganisms. The microorganism to be transformed for the purpose of expressing a variant CBH1 according to the present invention can be chosen from a wide variety of host cells. The sections below are provided as examples of host cells/microorganisms and are not meant to limit the scope of host cells that can be employed in practicing aspects of the present invention.

(i) Filamentous Fungi

Aspect of the present invention include filamentous fungi which have been modified, selected and cultured in a manner effective to result in desired CBH variant production or expression relative to the corresponding non-transformed parental filamentous fungi.

Examples of species of parental filamentous fungi that may be treated and/or modified for desired cellulase expression include, but are not limited to Trichoderma, Penicillium sp., Humicola sp., including Humicola insolens; Aspergillus sp., including Aspergillus niger, Chrysosporium sp., Fusarium sp., Hypocrea sp., and Emericella sp.

Cells expressing a desired CBH variant are cultured under conditions typically employed to culture the parental fungal line. Generally, cells are cultured in a standard medium containing physiological salts and nutrients, such as described in Pourquie, J. et al., Biochemistry and Genetics of Cellulose Degradation, eds. Aubert, J. P. et al., Academic Press, pp. 71-86, 1988 and Ilmen, M. et al., Appl. Environ. Microbiol. 63:1298-1306, 1997. Standard culture conditions are known in the art, e.g., cultures are incubated at 28° C. in shaker cultures or fermenters until desired levels of desired CBH variant expression are achieved.

Culture conditions for a given filamentous fungus can be found, for example, in the scientific literature and/or from the source of the fungi such as the American Type Culture Collection (ATCC). After fungal growth has been established, the cells are exposed to conditions effective to cause or permit the expression of a desired CBH variant.

In cases where a desired CBH variant coding sequence is under the control of an inducible promoter, the inducing agent, e.g., a sugar, metal salt or antibiotic, is added to the medium at a concentration effective to induce expression of the desired CBH variant.

In one embodiment, the strain is an Aspergillus niger strain, which is a useful strain for obtaining overexpressed polypeptide. For example A. niger var awamori dgr246 is known to secrete elevated amounts of secreted cellulases (Goedegebuur et al, Curr. Genet (2002) 41: 89-98). Other strains of Aspergillus niger var awamori such as GCDAP3, GCDAP4 and GAP3-4 are known Ward et al (Ward, M, Wilson, L. J. and Kodama, K. H., 1993, Appl. Microbiol. Biotechnol. 39:738-743).

In another embodiment, the strain is a Trichoderma reesei strain, which is a useful strain for obtaining overexpressed polypeptide. For example, RL-P37, described by Sheir-Neiss, et al., Appl. Microbiol. Biotechnol. 20:46-53 (1984) is known to secrete elevated amounts of cellulase enzymes. Functional equivalents of RL-P37 include Trichoderma reesei strain RUT-C30 (ATCC No. 56765) and strain QM9414 (ATCC No. 26921). It is contemplated that these strains would also be useful in overexpressing variant CBH.

Where it is desired to obtain the desired CBH variant in the absence of potentially detrimental native cellulase activity, it is useful to obtain a host cell strain which has had one or more cellulase genes deleted prior to introduction of a DNA construct or plasmid containing the DNA fragment encoding the desired CBH variant. Such strains may be prepared in any convenient manner, for example by the method disclosed in U.S. Pat. No. 5,246,853 and WO 92/06209, which disclosures are hereby incorporated by reference. By expressing a desired CBH variant in a host microorganism that is missing one or more cellulase genes (e.g., the endogenous CBH1 gene of a host cell), identification and subsequent purification procedures, where desired, are simplified.

Gene deletion may be accomplished by inserting a form of the desired gene to be deleted or disrupted into a plasmid by methods known in the art. The deletion plasmid is then cut at an appropriate restriction enzyme site(s), internal to the desired gene coding region, and the gene coding sequence or part thereof replaced with a selectable marker. Flanking DNA sequences from the locus of the gene to be deleted or disrupted, for example from about 0.5 to about 2.0 kb may remain on either side of the selectable marker gene. An appropriate deletion plasmid will generally have unique restriction enzyme sites present therein to enable the fragment containing the deleted gene, including flanking DNA sequences, and the selectable marker gene to be removed as a single linear piece.

In certain embodiments, more than one copy of DNA encoding a desired CBH variant may be present in a host strain to facilitate overexpression of the CBH variant. For example, a host cell may have multiple copies of a desired CBH variant integrated into the genome or, alternatively, include a plasmid vector that is capable of replicating autonomously in the host organism.

(ii) Yeast

The present invention also contemplates the use of yeast as a host cell for desired CBH production. Several other genes encoding hydrolytic enzymes have been expressed in various strains of the yeast S. cerevisiae. These include sequences encoding for two endoglucanases (Penttila et al., 1987), two cellobiohydrolases (Penttila et al., 1988) and one beta-glucosidase from Trichoderma reesei (Cummings and Fowler, 1996), a xylanase from Aureobasidlium pullulans (Li and Ljungdahl, 1996), an alpha-amylase from wheat (Rothstein et al., 1987), etc. In addition, a cellulase gene cassette encoding the Butyrivibrio fibrisolvens endo-[beta]-1,4-glucanase (END1), Phanerochaete chrysosporium cellobiohydrolase (CBH1), the Ruminococcus flavefaciens cellodextrinase (CEL1) and the Endomyces fibrilizer cellobiase (BgI1) was successfully expressed in a laboratory strain of S. cerevisiae (Van Rensburg et al., 1998).

(iii) Other

It is further contemplated that in some embodiments, expression systems in host cells other than filamentous fungal cells or yeast cells may be employed, including insect cell or bacterial cell expression systems. Certain of the bacterial host cells can, for example, be one that is also an ethanologen, such as an engineered Zymomonas mobilis, which is not only capable of expressing the enzyme(s)/variant(s) of interest but also capable of metabolizing certain monomeric and other fermentable sugars, turning them into ethanol. The selection of a host cell may be determined by the desires of the user of the CBH variants described herein, and thus no limitation in that regard is intended.

C. Introduction of a Desired CBH-Encoding Nucleic Acid Sequence into Host Cells.

The invention further provides cells and cell compositions which have been genetically modified to comprise an exogenously provided desired CBH variant-encoding nucleic acid sequence. A parental cell or cell line may be genetically modified (e.g., transduced, transformed or transfected) with a cloning vector or an expression vector. The vector may be, for example, in the form of a plasmid, a viral particle, a phage, etc., as further described above.

The methods of transformation of the present invention may result in the stable integration of all or part of the transformation vector into the genome of the host cell. However, transformation resulting in the maintenance of a self-replicating extra-chromosomal transformation vector is also contemplated.

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). In essence, the particular genetic engineering procedure used should be capable of successfully introducing a polynucleotide (e.g., an expression vector) into the host cell that is capable of expressing the desired CBH variant.

Many standard transfection methods can be used to produce Trichoderma reesei cell lines that express large quantities of the heterologus polypeptide. Some of the published methods for the introduction of DNA constructs into cellulase-producing strains of Trichoderma include Lorito, Hayes, DiPietro and Harman, 1993, Curr. Genet. 24: 349-356; Goldman, Van Montagu and Herrera-Estrella, 1990, Curr. Genet. 17:169-174; Penttila, Nevalainen, Ratto, Salminen and Knowles, 1987, Gene 6: 155-164, for Aspergillus Yelton, Hamer and Timberlake, 1984, Proc. Natl. Acad. Sci. USA 81: 1470-1474, for Fusarium Bajar, Podila and Kolattukudy, 1991, Proc. Natl. Acad. Sci. USA 88: 8202-8212, for Streptomyces Hopwood et al., 1985, The John Innes Foundation, Norwich, UK and for Bacillus Brigidi, DeRossi, Bertarini, Riccardi and Matteuzzi, 1990, FEMS Microbiol. Lett. 55: 135-138). An example of a suitable transformation process for Aspergillus sp. can be found in Campbell et al. Improved transformation efficiency of A. niger using homologous niaD gene for nitrate reductase. Curr. Genet. 16:53-56; 1989.

The invention further includes novel and useful transformants of host cells, e.g., filamentous fungi such as H. jecorina and A. niger, for use in producing fungal cellulase compositions. Thus, aspects of the subject invention include transformants of filamentous fungi comprising the desired CBH variant coding sequence, sometimes also including a deletion of the endogenous cbh coding sequence.

In addition, heterologous nucleic acid constructs comprising a desired cellulase-encoding nucleic acid sequence can be transcribed in vitro, and the resulting RNA introduced into the host cell by well-known methods, e.g., by injection.

D. Analysis for CBH1 Nucleic Acid Coding Sequences and/or Protein Expression.

In order to evaluate the expression of a desired CBH variant by a cell line that has been transformed with a desired CBH variant-encoding nucleic acid construct, assays can be carried out at the protein level, the RNA level or by use of functional bioassays particular to cellobiohydrolase activity and/or production.

In general, assays employed to analyze the expression of a desired CBH variant include, but are not limited to, Northern blotting, dot blotting (DNA or RNA analysis), RT-PCR (reverse transcriptase polymerase chain reaction), or in situ hybridization, using an appropriately labeled probe (based on the nucleic acid coding sequence) and conventional Southern blotting and autoradiography.

In addition, the production and/or expression of a desired CBH variant may be measured in a sample directly, for example, by assays for cellobiohydrolase activity, expression and/or production. Such assays are described, for example, in Becker et al., Biochem J. (2001) 356:19-30 and Mitsuishi et al., FEBS (1990) 275:135-138, each of which is expressly incorporated by reference herein. The ability of CBH1 to hydrolyze isolated soluble and insoluble substrates can be measured using assays described in Srisodsuk et al., J. Biotech. (1997) 57:49-57 and Nidetzky and Claeyssens Biotech. Bioeng. (1994) 44:961-966. Substrates useful for assaying cellobiohydrolase, endoglucanase or β-glucosidase activities include crystalline cellulose, filter paper, phosphoric acid swollen cellulose, cellooligosaccharides, methylumbelliferyl lactoside, methylumbelliferyl cellobioside, orthonitrophenyl lactoside, paranitrophenyl lactoside, orthonitrophenyl cellobioside, paranitrophenyl cellobioside.

In addition, protein expression, may be evaluated by immunological methods, such as ELISA, competitive immunoassays, radioimmunoassays, Western blot, indirect immunofluorescent assays, and the like. Certain of these assays can be performed using commercially available reagents and/or kits designed for detecting CBH enzymes. Such immunoassays can be used to qualitatively and/or quantitatively evaluate expression of a desired CBH variant. The details of such methods are known to those of skill in the art and many reagents for practicing such methods are commercially available. In certain embodiments, an immunological reagent that is specific for a desired variant CBH enzyme but not its parent CBH may be employed, e.g., an antibody that is specific for a CBH substitution or a fusion partner of the CBH variant (e.g., an N or C terminal tag sequence, e.g., a hexa-Histidine tag or a FLAG tag). Thus, aspects of the present invention include using a purified form of a desired CBH variant to produce either monoclonal or polyclonal antibodies specific to the expressed polypeptide for use in various immunoassays. (See, e.g., Hu et al., 1991).

### V. Methods for Enrichment, Isolation and/or Purification of CBH Variant Polypeptide

In general, a desired CBH variant polypeptide produced in a host cell culture is secreted into the medium (producing a culture supernatant containing the CBH variant) and may be enriched, purified or isolated, e.g., by removing unwanted components from the cell culture medium. However, in some cases, a desired CBH variant polypeptide may be produced in a cellular form necessitating recovery from a cell lysate. The desired CBH variant polypeptide is harvested from the cells or cell supernatants in which it was produced using techniques routinely employed by those of skill in the art. Examples include, but are not limited to, filtration (e.g., ultra- or micro-filtration), centrifugation, density gradient fractionation (e.g., density gradient ultracentrifugation), affinity chromatography (Tilbeurgh et al., 1984), ion-exchange chromatographic methods (Goyal et al., 1991; Fliess et al., 1983; Bhikhabhai et al., 1984; Ellouz et al., 1987), including ion-exchange using materials with high resolution power (Medve et al., 1998), hydrophobic interaction chromatography (Tomaz and Queiroz, 1999), and two-phase partitioning (Brumbauer, et al., 1999).

While enriched, isolated or purified CBH variant polypeptide is sometimes desired, in some embodiments, a host cell expressing a CBH variant polypeptide is employed directly in an assay that requires cellobiohydrolase activity. Thus, enrichment, isolation or purification of the desired CBH variant polypeptide is not always required to obtain a CBH variant polypeptide composition that finds use in a cellulase assay or process. For example, a cellulase system according to aspects of the present invention might be designed to allow a host cell that expresses a variant CBH1 as described herein to be used directly in a cellulase process, i.e., without isolation of the CBH1 away from the host cell prior to its use in an assay of interest. In one such example, CBH1 variant-expressing yeast cells may be added directly into a fermentation process such that the yeast cell expresses the variant CBH1 directly into the fermentation broth where its cellulase activity converts a non-fermentable substrate into fermentable sugars for the yeast cell to convert directly to a desired product, e.g., into ethanol (see, e.g., Ilmén et al., High level secretion of cellobiohydrolases by Saccharomyces cerevisiae Biotechnology for Biofuels 2011, 4:30).

### VI. Utility of CBH1 Variants

It can be appreciated that the desired CBH variant-encoding nucleic acids, the desired CBH variant polypeptide and compositions comprising the same find utility in a wide variety applications, some of which are described below. The improved property or properties of the CBH variants described herein can be exploited in many ways. For example, CBH variants with improved performance under conditions of thermal stress can be used to increase cellulase activity in assays carried out at high temperatures (e.g., temperatures at which the parent CBH would perform poorly), allowing a user to reduce the total amount of CBH employed (as compared to using the parent CBH). Other improved properties of CBH variant polypeptides can be exploited in cellulase assays, including CBH variants having altered pH optima, increased stability or activity in the presence of surfactants, increased specific activity for a substrate, altered substrate cleavage pattern, and/or high level expression in a host cell of interest.

Thus, CBH variant polypeptides as describe herein find use in detergent compositions that exhibit enhanced cleaning ability, function as a softening agent and/or improve the feel of cotton fabrics (e.g., “stone washing” or “biopolishing”), in compositions for degrading wood pulp into sugars (e.g., for bio-ethanol production), and/or in feed compositions. The isolation and characterization of CBH variants provides the ability to control characteristics and activity of such compositions.

A cellulase composition containing a desired CBH variant as described herein finds use in ethanol production. Ethanol from this process can be further used as an octane enhancer or directly as a fuel in lieu of gasoline which is advantageous because ethanol as a fuel source is more environmentally friendly than petroleum derived products. It is known that the use of ethanol will improve air quality and possibly reduce local ozone levels and smog. Moreover, utilization of ethanol in lieu of gasoline can be of strategic importance in buffering the impact of sudden shifts in non-renewable energy and petro-chemical supplies.

Separate saccharification and fermentation is a process whereby cellulose present in biomass, e.g., corn stover, is converted to glucose and subsequently yeast strains convert the glucose into ethanol. Simultaneous saccharification and fermentation is a process whereby cellulose present in biomass is converted to glucose and, at the same time and in the same reactor, yeast strains convert glucose into ethanol. Thus, the CBH variants of the invention find use in the both of these processes for the degradation of biomass to ethanol. Ethanol production from readily available sources of cellulose provides a stable, renewable fuel source. It is further noted that in some processes, biomass is not fully broken down to glucose (containing, e.g., disaccharides), as such products find uses apart from ethanol production.

Cellulose-based feedstocks can take a variety of forms and can contain agricultural wastes, grasses and woods and other low-value biomass such as municipal waste (e.g., recycled paper, yard clippings, etc.). Ethanol may be produced from the fermentation of any of these cellulosic feedstocks. As such, a large variety of feedstocks may be used with the inventive desired cellulase(s) and the one selected for use may depend on the region where the conversion is being done. For example, in the Midwestern United States agricultural wastes such as wheat straw, corn stover and bagasse may predominate while in California rice straw may predominate. However, it should be understood that any available cellulosic biomass may be used in any region.

In another embodiment the cellulosic feedstock may be pretreated. Pretreatment may be by elevated temperature and the addition of dilute acid, concentrated acid or dilute alkali solution. The pretreatment solution is added for a time sufficient to at least partially hydrolyze the hemicellulose components and then neutralized.

In addition to biomass conversion, CBH variant polypeptides as described herein can be present in detergent compositions which can include any one or more detergent components, e.g., a surfactant (including anionic, non-ionic and ampholytic surfactants), a hydrolase, building agents, bleaching agents, bluing agents and fluorescent dyes, caking inhibitors, solubilizers, cationic surfactants and the like. All of these components are known in the detergent art. The CBH variant polypeptide-containing detergent composition can be in any convenient form, including liquid, granule, emulsion, gel, paste, and the like. In certain forms (e.g., granules) the detergent composition can be formulated so as to contain a cellulase protecting agent. For a more thorough discussion, see U.S. Pat. No. 6,162,782 entitled “Detergent compositions containing cellulase compositions deficient in CBH1 type components,” which is incorporated herein by reference.

In certain embodiments, the CBH variant polypeptide is present in the detergent compositions from 0.00005 weight percent to 5 weight percent relative to the total detergent composition, e.g., from about 0.0002 weight percent to about 2 weight percent relative to the total detergent composition.

It is noted that CBH variants with decreased thermostability find use, for example, in areas where the enzyme activity is required to be neutralized at lower temperatures so that other enzymes that may be present are left unaffected. In addition, the enzymes may find utility in the limited conversion of cellulosics, for example, in controlling the degree of crystallinity or of cellulosic chain-length. After reaching the desired extent of conversion, the saccharifying temperature can be raised above the survival temperature of the de-stabilized CBH variant. As the CBH activity is essential for hydrolysis of crystalline cellulose, conversion of crystalline cellulose will cease at the elevated temperature.

As seen from above, CBH variant polypeptides (and the nucleic acids encoding them) with improved properties as compared to their parent CBH enzymes find use in improving any of a number of assays and processes that employ cellobiohydrolases.

### EXAMPLES

The present invention is described in further detain in the following examples which are not in any way intended to limit the scope of the invention as claimed. The attached Figures are meant to be considered as integral parts of the specification and description of the invention. All references cited are herein specifically incorporated by reference for all that is described therein.

### Example 1

### I. Assays

The following assays were used in the examples described below. Any deviations from the protocols provided below are indicated in the examples. In these experiments, a spectrophotometer was used to measure the absorbance of the products formed after the completion of the reactions.

### A. Performance Index

The performance index (P1) compares the performance or stability of the variant (measured value) and the standard enzyme (theoretical value) at the same polypeptide concentration. In addition, the theoretical values can be calculated using the parameters of the Langmuir equation of the standard enzyme. A dose response curve was generated for the wild-type EG4 by fitting the data with the Langmuir equation with intercept (y=((x*a)/(x+b))+c) and the activities of the EG4 variants were divided by a calculated activity of wild-type EG4 of the same plate to yield a performance index. A performance index (P1) that is greater than 1 (PI>1) indicates improved performance by a variant as compared to the standard (e.g., wild-type Hypocrea jecorina cellobiohydrolase 1, also known as CBH1 or Cel7A), while a PI of 1 (PI=1) identifies a variant that performs the same as the standard, and a PI that is less than 1 (PI<1) identifies a variant that performs worse than the standard.

B. Protein Content Determination

The concentration of CBH1 variant polypeptides from pooled culture supernatants was determined using an Agilent 1200 HPLC equipped with a Acquity UPLC BEH200 SEC 1.7 μm (4.6×150 mm) column (Waters #186005225). Twenty five (25) microliters of sample was mixed with 75 μL of de-mineralized water. Ten (10) μL of the 4× diluted sample was injected onto the column. To elute the sample, 25 mM NaH2PO4 pH6.7+100 mM NaCl was run isocratically for 5.0 min. Protein concentrations of CBH1 variants were determined from a calibration curve generated using purified wild-type CBH1 (0-1410 ppm). To calculate performance index (P, or PI), the ratio of the (average) total protein produced by a variant and (average) total protein produced by the wild-type at the same dose were averaged.

### C. ABTS Assay for Measurement of Glucose

Residual glucose from H. jecorina culture supernatants expressing CBH1 variants was measured. Supernatants of cultures with residual glucose were excluded from pooling for further studies. Monomeric glucose was detected using the ABTS assay. The assay buffer contained 2.74 g/L 2,2′-azino-bis(3-ethylbenzo-thiazoline-6-sulfonic acid) di-ammonium salt (ABTS, Sigma, catalog no. A1888), 0.1 U/mL horseradish peroxidase Type VI-A (Sigma, catalog no. P8375), and 1 Unit/mL food grade glucose oxidase (GENENCOR® 5989 U/mL) in 50 mM sodium acetate buffer pH 5.0. Ten (10) microliters (diluted) BGL1 activity assay mix was added to 100 μL ABTS assay solution. After adding the activity assay mix, the reaction was followed kinetically for 5 min at OD₄₂₀, at ambient temperature of 22° C. An appropriate calibration curve of glucose for each assay condition was always included.

### D. Phosphoric Acid Swollen Cellulose (PASC) Hydrolysis Assays

### D.1. Phosphoric Acid Swollen Cellulose (PASC) Hydrolysis Assay

Phosphoric acid swollen cellulose (PASO) was prepared from Avicel according to a published method (Walseth, Tappi 35:228, 1971; and Wood, Biochem J, 121:353-362, 1971). This material was diluted with buffer and water to achieve a 0.5% w/v mixture such that the final concentration of sodium acetate was 50 mM, pH 5.0. CBH1 activity was determined by adding 15 μL culture supernatant to 85 μL reaction mix (0.15% PASO; 0.42 mg/ml culture supernatant of a H. jecorina strain deleted for cbh1, cbh2, eg1, eg2, eg3, and bgl1; 29.4 mM NaOAc (pH5.0)) in a 96-well microtiterplate (Costar Flat Bottom PS 3641). The micro-titer plate was sealed and incubated in a thermostatted incubator at 50° C. under continuous shaking at 900 rpm for 3 hours, followed by 5 min cooling on ice. The hydrolysis reaction was stopped by the addition of 100 μL quench buffer (100 mM glycine buffer (pH 10); 5 mg/ml calcofluor (Sigma)). Activity was determined according to a published method (Du et al, Appl Biochem Biotechnol 161(1-8): 313-7). A dose response curve was generated for wild-type CBH1 enzyme. Assays were performed in quadruplicate. To calculate performance index (P, or PI), the ratio of the (average) total sugar produced by a variant and (average) total sugar produced by the wild-type at the same dose were averaged.

### D.2. Phosphoric Acid Swollen Cellulose (PASC) Hydrolysis Assay

Phosphoric acid swollen cellulose (PASO) was prepared from Avicel according to a published method (Walseth, Tappi 35:228, 1971; and Wood, Biochem J, 121:353-362, 1971). This material was diluted with buffer and water to achieve a 0.5% w/v mixture such that the final concentration of sodium acetate was 50 mM, pH 5.0. CBH1 activity was determined by adding 5 μL, 10 μL, 20 μL and 40 μL of 400 ppm anion purified (see I.1) CBH1 to 140 μL reaction mix (0.36% PASO; 29.4 mM NaOAc (pH 5.0); 143 mM NaCl) in a 96-well microtiterplate (Costar Flat Bottom PS 3641). The micro-titer plate was sealed and incubated in a thermostatted incubator at 50° C. under continuous shaking at 900 rpm for 2 hours, followed by 5 min cooling on ice. The hydrolysis reaction was stopped by the addition of 100 μL quench buffer (100 mM glycine buffer (pH 10). The hydrolysis reaction products were analyzed with a PAHBAH assay according to Lever, 1972, Anal Biochem, 47:273-279 with the following modifications: PAHBAH assay: Aliquots of 150 μL of PAHBAH reducing sugar reagent (for 100 mL reagent: 1.5 g p-hydroxybenzoic acid hydrazide (Sigma # H9882), 5 g Potassium sodium tartrate tetrahydrate dissolved in 2% NaOH), were added to all wells of an empty microtiter plate. Ten (10) microliters of the hydrolysis reaction supernatants were added to the PABAH reaction plate. All plates were sealed and incubated at 69° C. under continuous shaking of 900 rpm. After one hour the plates were placed on ice for five minutes and centrifuged at 720×g at room temperature for five minutes. Absorbance of plates (endpoint) was measured at 410 nm in a spectrophotometer. A cellobiose standard was included as control and appropriate blank samples. A dose response curve was generated for wild-type CBH1 enzyme. To calculate performance index (PI), the (average) total sugar produced by a variant CBH1 was divided by the (average) total sugar produced by the wild-type CBH1 (e.g. a reference enzyme) at the same dose.

### D.3. Phosphoric Acid Swollen Cellulose (PASC) Hydrolysis Assay in the Presence of EGII

The PASO assay in the presence of 2.5 ppm T. reesei EGII was performed as described for the assay under D.2 (i.e., without EGII) with the following modifications: 400 ppm of anion purified CBH1 enzyme was diluted 1.6 fold before addition to the assay, reaction additions was the same as under D.2 only 10 μL of 37.5 ppm EGII was added to the reaction mix resulting in a total reaction volume of 150 μL. PI was calculated as described under D.2.

E. Whole Hydrolysate Acid-Pretreated Corn Stover (whPCS) Assay

Corn stover was pretreated with 2% w/w H₂SO₄ as described (Schell et al., J Appl Biochem Biotechnol, 105:69-86, 2003). Volumes of 3, 5, 10 and 25 μL supernatant (2-fold diluted in 50 mM NaOAc) were added to whPCS reaction mixtures (6.5% (w/v) whPCS; 1.43 mg/ml supernatant of H. jecorina deleted for cbh1 and cbh2 (as described in WO 2005/001036); 0.22 mg/ml Xyn3; 0.15 mg/ml Fv51A; 0.18 mg/ml Fv3A; 0.15 mg/ml Fv43D; 0.22 mg/ml BGL1 with a final total volume of 160 μL. (Examples of suitable methods employing the enzymes Xyn3, Fv51A, Fv3A, Fv43D, and Bgl1 are described in PCT application publication WO2011/0038019). The micro-titer plate was sealed and incubated in a thermostatted incubator at 50° C. under continuous shaking at 900 rpm for 3 hours, followed by 5 min cooling on ice. The hydrolysis reaction was stopped by the addition of 100 μL quench buffer (100 mM glycine buffer, pH 10). Plates were centrifuged at room temperature for 5 minutes at 3,000 rpm, and a 20× dilution of the sample was made by adding 10 μL of the sample to 190 μL of water. Free glucose in the reaction was measured using the ABTS assay as described under assay C.

F. Dilute Ammonia Corn Stover (daCS) Assay

Dilute ammonia pretreated corn stover was prepared essentially as described for dilute ammonia corncob (WO2006/110901). Pretreated corn stover was used as a 10% cellulose suspension in 50 mM sodium acetate (pH 5.0). Volumes of 3, 5, 10 and 20 μL supernatant were added to daCS reaction mixtures (5.8% (w/v) cellulose; 0.052 mg/ml H. jecorina CBH2; 0.13 mg/ml H. jecorina Xyn3; 0.011 mg/ml Fv51A; 0.006 mg/ml Fv3A; 0.011 mg/ml Fv43D; 0.08 mg/ml Fv3C; 0.04 mg/ml EG4; 0.05 mg/ml H. jecorina Δ(cbh1, cbh2) with a final total volume of 120 μL. (As noted above, examples of suitable methods employing the enzymes Xyn3, Fv51A, Fv3A, Fv43D, and Fv3C are described in PCT application publication WO2011/0038019). The micro-titer plate was sealed and incubated in a thermostatted incubator at 50° C. under continuous shaking at 900 rpm for 24 hours, followed by 5 min cooling on ice. The hydrolysis reaction was stopped by the addition of 100 μL quench buffer (100 mM glycine buffer (pH 10). Plates were centrifuged at room temperature for 5 minutes at 3,000 rpm, and a 20× dilution of the sample was made by adding 10 μL of the sample to 190 μL of water. Free glucose in the reaction was measured using the ABTS assay as described under assay C.

### G. Protein Purification

For micro-scale purification, 200 μL of 90% ethanol was transferred to a Multiscreen deep-well solvinert hydrophobic PTFE filter plate (MiliPore #MDRPN0410) followed by 1 min centrifugation at 50×g. Four hundred (400) μL of DEAE Sepharose Fast-Flow resin (GE-Healthcare #17-0709-01) was transferred to the filter plate followed by centrifugation of 1 min at 50×g. The resin was washed three times using 400 μL MiliQ water, and equilibrated three times using 400 μL of 25 mM NaH₂PO₄ (pH6.7). Four hundred and fifty (450) μL of culture supernatant was diluted 6× to 2700 μL using 25 mM NaH₂PO₄ (pH6.7). Diluted samples were loaded on the resin. To elute all unbound protein, the resin was washed three times with 25 mM NaH₂PO₄ (pH6.7). CBH1 variants were eluted using 400 μL of 25 mM NaAc pH5.0+500 mM NaCl.

For large-scale purification, a Vivaspin20 10kDMWO filter (Sartorius #VS2001) was used to concentrate 20 mL of CBH1 shake flask sample to 2.5 mL (centrifuged for 20 minutes at 3000×g). The concentrated sample was diluted to 10 mL using 50 mM NaAc pH5.0. A 1 mL Hitrap DEAE FF column (GE-Healthcare #17-5055-01) was equilibrated using 25 mM NaAc pH5.0. The diluted sample was loaded on the column at 1.0 mL/min. After complete loading of the sample, the column was washed with 12 column volumes (CV) of 25 mM NaAc pH5.0 at 1 mL/min. CBH1 was eluted from the column using a 30 CV gradient from 0% to 50% of 25 mM NaAc pH5.0+1M NaCl. During the gradient, fractions of 5 mL were collected. Fractions were analyzed by SDS-PAGE. The three fractions containing most CBH1 were pooled.

### H. Measurement of Protein Melting Temperature (Tm)

Stability of CBH1 variants was determined by a fluorescent dye-binding thermal shift assay (Lavinder et al, High-throughput thermal scanning: A general, rapid dye-binding thermal shift screen for protein engineering (2009) JACS, 131: 3794-3795). SyproOrange (Molecular Probes) was diluted 1:1000 in MQ water. In a well, 8 μl diluted dye was mixed with 25 μl 100 mg/l enzyme in 50 mM NaOAc (pH5). Sealed plates were subjected to a temperature gradient of 25° C. to 95° C. with an approximate rate of 1° C./min in an ABI 7900HT rtPCR system (Applied Biosystems). The mid-peak temperature of the first derivative of the fluorescence signal was taken as the melting temperature (Tm) of the CBH1 enzyme in the sample.

### Example 2

### Generation of Hypocrea jecorina CBH1 Variants

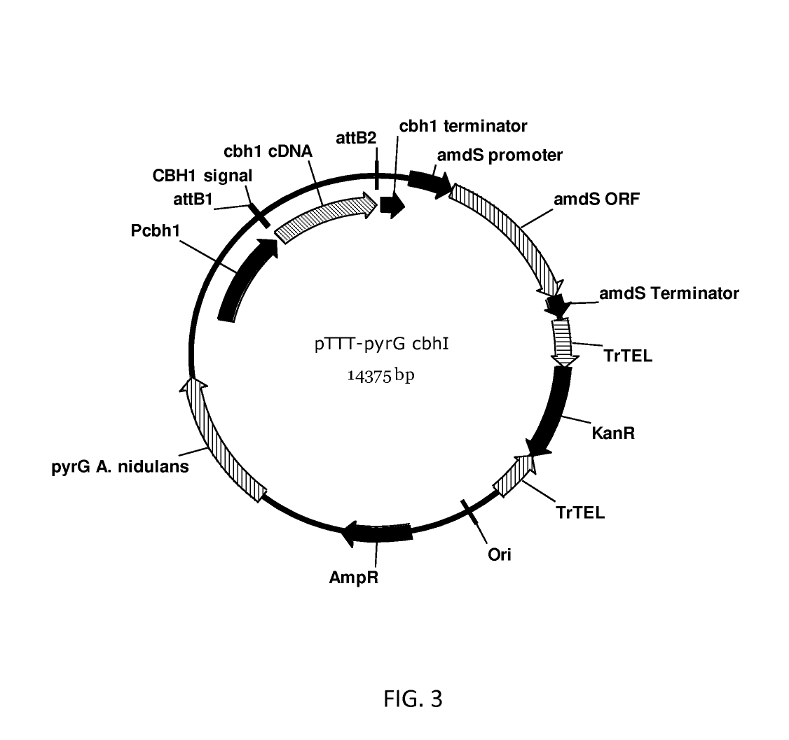

In this example, the construction of Trichoderma reesei strains expressing wild-type Hypocrea jecorina cellobiohydrolase 1 (CBH1) and variants, thereof, are described. A cDNA fragment listed below as SEQ ID NO: 1 (previously described in U.S. Pat. No. 7,452,707), encoding CBH1 (SEQ ID NO: 3) served as template DNA for the construction of Trichoderma reesei strains expressing CBH1 and variants thereof. The cDNA was inserted into the expression plasmid pTTT-pyrG to generate pTTT-pyrG-cbh1 (as shown in FIG. 3).

SEQ ID NO: 1 includes the wild type nucleotide sequence encoding the mature form of H. jecorina cbh1 adjacent to a sequence encoding the CBH1 signal peptide (underlined):

atgtatcggaagttggccgtcatctcggccttcttggccacagctcg
tgctcagtcggcctgcactctccaatcggagactcacccgcctctga
catggcagaaatgctcgtctggtggcacgtgcactcaacagacaggc
tccgtggtcatcgacgccaactggcgctggactcacgctacgaacag
cagcacgaactgctacgatggcaacacttggagctcgaccctatgtc
ctgacaacgagacctgcgcgaagaactgctgtctggacggtgccgcc
tacgcgtccacgtacggagttaccacgagcggtaacagcctctccat
tggctttgtcacccagtctgcgcagaagaacgttggcgctcgccttt
accttatggcgagcgacacgacctaccaggaattcaccctgcttggc
aacgagttctctttcgatgttgatgtttcgcagctgccgtgcggctt
gaacggagctctctacttcgtgtccatggacgcggatggtggcgtga
gcaagtatcccaccaacaccgctggcgccaagtacggcacggggtac
tgtgacagccagtgtccccgcgatctgaagttcatcaatggccaggc
caacgttgagggctgggagccgtcatccaacaacgcgaacacgggca
ttggaggacacggaagctgctgctctgagatggatatctgggaggcc
aactccatctccgaggctcttaccccccacccttgcacgactgtcgg
ccaggagatctgcgagggtgatgggtgcggcggaacttactccgata
acagatatggcggcacttgcgatcccgatggctgcgactggaaccca
taccgcctgggcaacaccagcttctacggccctggctcaagctttac
cctcgataccaccaagaaattgaccgttgtcacccagttcgagacgt
cgggtgccatcaaccgatactatgtccagaatggcgtcactttccag
cagcccaacgccgagcttggtagttactctggcaacgagctcaacga
tgattactgcacagctgaggaggcagaattcggcggatcctctttct
cagacaagggcggcctgactcagttcaagaaggctacctctggcggc
atggttctggtcatgagtctgtgggatgattactacgccaacatgct
gtggctggactccacctacccgacaaacgagacctcctccacacccg
gtgccgtgcgcggaagctgctccaccagctccggtgtccctgctcag
gtcgaatctcagtctcccaacgccaaggtcaccttctccaacatcaa
gttcggacccattggcagcaccggcaaccctagcggcggcaaccctc
ccggcggaaacccgcctggcaccaccaccacccgccgcccagccact
accactggaagctctcccggacctacccagtctcactacggccagtg
cggcggtattggctacagcggccccacggtctgcgccagcggcacaa
cttgccaggtcctgaacccttactactctcagtgcctg

SEQ ID NO:2 sets forth the sequence of the H. jecorina CBH1 full length polypeptide containing the CBH1 signal peptide (underlined):

Myrklavisaflataraqsactlqsethppltwqkcssggtctqqtg
svvidanwrwthatnsstncydgntwsstlcpdnetcaknccldgaa
yastygvttsgnslsigfvtqsaqknvgarlylmasdttyqeftllg
nefsfdvdvsqlpcglngalyfvsmdadggvskyptntagakygtgy
cdsqcprdlkfingqanvegwepssnnantgigghgsccsemdiwea
nsisealtphpcttvgqeicegdgcggtysdnryggtcdpdgcdwnp
yrlgntsfygpgssftldttkkltvvtqfetsgainryyvqngvtfq
qpnaelgsysgnelnddyctaeeaefggssfsdkggltqfkkatsgg
mvlvmslwddyyanmlwldstyptnetsstpgavrgscstssgvpaq
vesqspnakvtfsnikfgpigstgnpsggnppggnppgttttrrpat
ttgsspgptqshygqcggigysgptvcasgttcqvlnpyysqcl

SEQ ID NO:3 sets forth the sequence of the H. jecorina CBH1 mature polypeptide:

Qsactlqsethppltwqkcssggtctqqtgsvvidanwrwthatnss
tncydgntwsstlcpdnetcaknccldgaayastygvttsgnslsig
fvtqsaqknvgarlylmasdttyqeftllgnefsfdvdvsqlpcgln
galyfvsmdadggvskyptntagakygtgycdsqcprdlkfingqan
vegwepssnnantgigghgsccsemdiweansisealtphpcttvgq
eicegdgcggtysdnryggtcdpdgcdwnpyrlgntsfygpgssftl
dttkkltvvtqfetsgainryyvqngvtfqqpnaelgsysgnelndd
yctaeeaefggssfsdkggltqfkkatsggmvlvmslwddyyanmlw
ldstyptnetsstpgavrgscstssgvpaqvesqspnakvtfsnikf
gpigstgnpsggnppggnppgttttrrpatttgsspgptqshygqcg
gigysgptvcasgttcqvlnpyysqcl

The pTTTpyrG-cbh1 plasmid containing the Hypocrea jecorina CBH1 enzyme encoding sequence (SEQ ID NO: 1) was used as a template to generate CBH1 variants.

### Production of CBH1 Variant Polypeptides

Purified pTTTpyrG-cbh1 plasmids (P_{cbh1}, Amp^{R}, acetamidase; see plasmid schematic shown in FIG. 3) expressing genes encoding CBH1 variant enzymes were expressed in a six gene deleted Trichoderma reesei strain (Δegl1, Δegl2, Δegl3, Δcbh1, Δcbh2, Δbgl1) that was derived from RL-P37 (Sheir-Neiss, G et al. Appl. Microbiol. Biotechnol. 1984, 20:46-53), and is further described in PCT Application Publication WO2010/141779. Gene deletions were created according to the methods described in PCT Application Publication WO2005/001036 for making a four gene deleted T. reesei strain (Δegl1, Δegl2, Δcbh1, Δcbh2), which was similarly further deleted for egl3 and bgl1, resulting in the six gene deleted strain. Protoplasts of the six-fold deleted T. reesei were transformed with the individual pTTT-pyrG-cbh1 constructs (a single CBH1 variant per transformation) and grown on selective agar containing acetamide at 28° C. for 7 d as previously described in PCT Application Publication WO2009/048488. Transformants of T. reesei were revived on selective agar containing acetamide and incubated at 28° C. for 7 d. Spores were harvested by scraping each well with 300 μL saline+0.015% Tween-80. For CBH1 variant production, a volume of 10 μL or 25 μL spore suspension was added to 200 μL of 1 mL Aachen medium in a 96-well or 24-well plate respectively. The plates were closed with an Enzyscreen lid and fermented for 7 days at 28° C. and 80% humidity in a 50 mm throw Infors incubator. The broth was transferred to 96-well filterplates and filtrated under vacuum. Residual glucose was measured using the ABTS assay as described in Example C. The remaining spore suspensions were stored in 50% glycerol at −80° C.

### Example 3

### CBH1 Variants with Significant Benefit to Tm

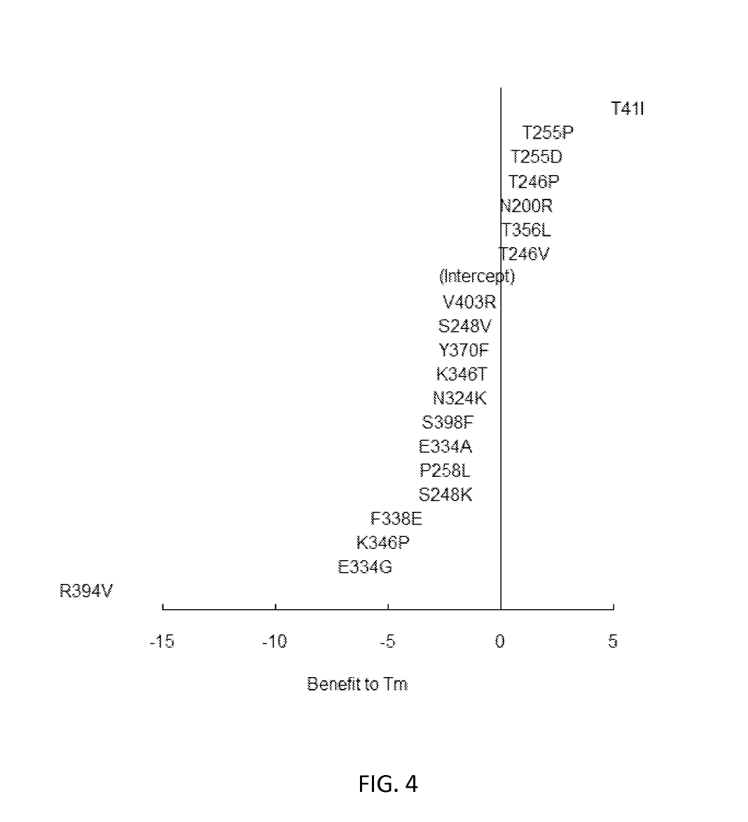

The Tm for the CBH1 variants, including multiply substituted variants, were determined as described above in H and analyzed to model how each specific substitution affected Tm. The substitutions that display significant changes to Tm (significance to 0.001, or 99.9%) are shown in the graph in FIG. 4 and Table 1. In FIG. 4, the change to Tm is on the X axis with each specific variant modeled shown at its change to Tm value, which can be positive or negative. The “intercept” value indicates the model's prediction of change to Tm for a molecule with no substitutions (i.e., wild type CBH1). (Note that the model's prediction is not always 0).

As shown in FIG. 4 and Table 1 (which shows the modeled change to Tm value for each CBH1 variant in FIG. 4; numbers in parentheses are negative), the following variants significantly increased Tm (i.e., significantly greater than 0): T41I, T255P, T255D, T246P, N200R, T356L, and T246V.

As shown in FIG. 4 and Table 1, the following variants significantly reduced Tm (i.e., significantly less than 0): V403R, S248V, Y370F, K346T, N324K, S398F, E334A, P258L, S248K, F338E, K346P, E334G, and R394V.

It is noted hear that while in certain embodiments, CBH1 variants having significantly increased Tm are desired, e.g., for use in processes in which resistance to high temperature inactivation of the polypeptide are desired, in other embodiments, CBH1 variants having significantly decreased Tm are desired, e.g., for use in processes in which a high temperature CBH1 inactivation step is desired. As such, the desirability of the CBH1 variants shown in FIG. 4 and Table 1 depends on the intended use of the variant.

TABLE 1
CBH1 Variants Having Significant Tm values

| Variant | ΔTm value |
|---|---|
| T41I | 5.7 |
| T255P | 2.1 |
| T255D | 1.6 |
| T246P | 1.5 |
| N200R | 1.2 |
| T356L | 1.2 |
| T246V | 1.1 |
| INTERCEPT | (1.0)* |
| V403R | (1.3) |
| S248V | (1.5) |
| Y370F | (1.6) |
| K346T | (1.7) |
| N324K | (1.8) |
| S398F | (2.3) |
| E334A | (2.4) |
| P258L | (2.4) |
| S248K | (2.4) |
| F338E | (4.6) |
| K346P | (5.2) |
| E334G | (6.0) |
| R394V | (18.3) |

*Numbers in parentheses are negative.

### Example 4

### CBH1 Variants with Significant Benefit in whPCS PI Assay

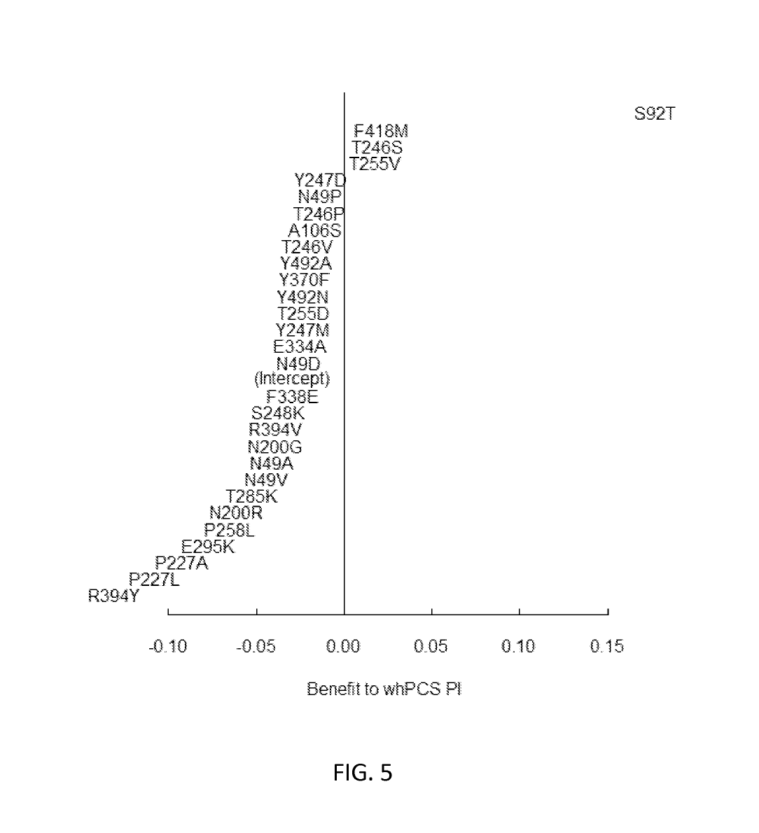

The performance index (PI) for the CBH1 variants, including multiply substituted variants, were determined as described above in E and analyzed to model how each specific substitution significantly affected the PI (significance to 0.10, or 90%). The substitutions that display significant changes in PI are shown in the graph in FIG. 5 and Table 2. In FIG. 5, the change in PI (or ΔPI value) is on the X axis (labeled “Benefit to whPCS PI”) with each specific variant having significant change in PI shown at its approximate ΔPI value. The “intercept” value indicates the model's prediction of change in PI for a molecule with no substitutions (i.e., wild type CBH1). (Note that the model's prediction is not always 0).

As shown in FIG. 5 and Table 2 (which shows the ΔPI value for each CBH1 variant in FIG. 5; numbers in parentheses are negative), the following variants displayed a significantly increased PI (i.e., significantly greater than 0): S92T, F418M, T246S, and T255V.

As shown in FIG. 5 and Table 2, the following variants displayed a significantly reduced PI (i.e., significantly less than 0): Y247D*, N49P*, T246P*, A106S*, T246V*, Y492A*, Y370F*, Y492N*, T255D*, Y247M*, E334A*, N49D*, S248K, R394V, N200G, N49A, N49V, T285K, N200R, P258L, E295K, P227A, P227L, and R394Y. The variants indicated with * had a modeled ΔPI that is significantly less than 0 but greater than the modeled ΔPI for the intercept (i.e., wild type).

TABLE 2
CBH1 Variants Having Significant ΔPI values in whPCS Assay

| Variant | ΔPI value |
|---|---|
| S92T | 0.18 |
| F418M | 0.02 |
| T246S | 0.02 |
| T255V | 0.018 |
| Y247D | (0.013)* |
| N49P | (0.013) |
| T246P | (0.014) |
| A106S | (0.016) |
| T246V | (0.021) |
| Y492A | (0.021) |
| Y370F | (0.022) |
| Y492N | (0.023) |
| T255D | (0.023) |
| Y247M | (0.023) |
| E334A | (0.025) |
| N49D | (0.025) |
| INTERCEPT | (0.029) |
| F338E | (0.029) |
| S248K | (0.038) |
| R394V | (0.038) |
| N200G | (0.039) |
| N49A | (0.041) |
| N49V | (0.044) |
| T285K | (0.052) |
| N200R | (0.061) |
| P258L | (0.064) |
| E295K | (0.077) |
| P227A | (0.092) |
| P227L | (0.11) |
| R394Y | (0.13) |

*Numbers in parentheses are negative.

### Example 5

### CBH1 Variants with Significant Benefit in daCS PI Assay

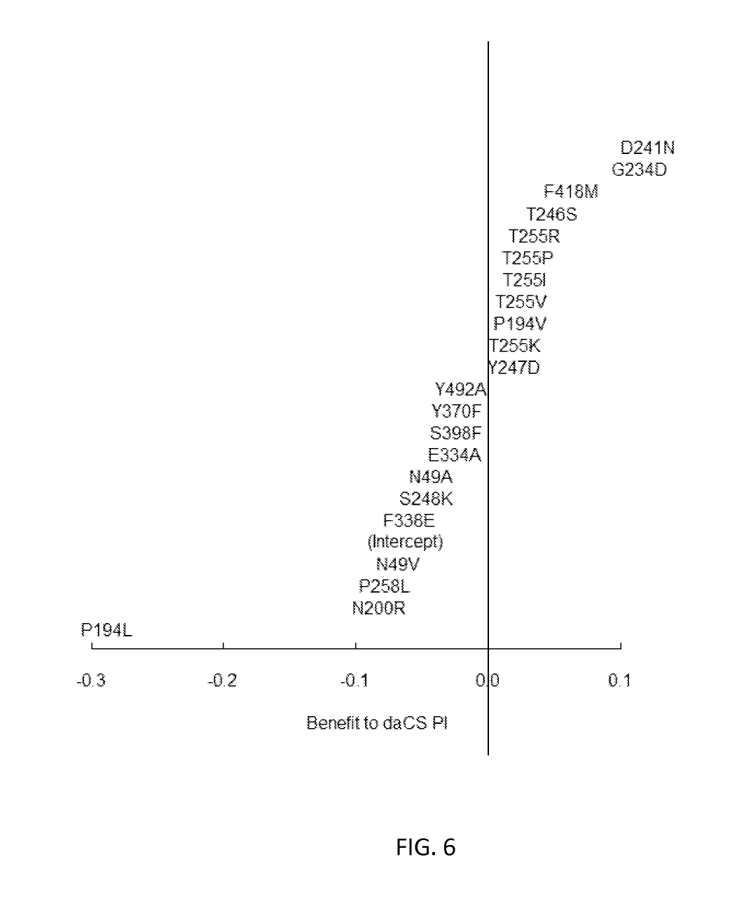

The performance index (PI) was determined for individually substituted CBH1 variants in daCS assays as described above in F and analyzed to determine whether the PI as compared to the wild type CBH1 enzyme was significantly reduced or significantly increased (significance to 0.25, or 75%). The substitutions that display significant changes in PI are shown in the graph in FIG. 6 and in Table 3. In FIG. 6, the change in PI (or ΔPI value) is on the X axis (labeled “Benefit to daCS PI”) with each specific variant having significant change in PI shown at its approximate ΔPI value. The “intercept” value indicates the model's prediction of change in PI for a molecule with no substitutions (i.e., wild type CBH1). (Note that the model's prediction is not always 0).

As shown in FIG. 6 and Table 3 (which shows the ΔPI value for each CBH1 variant in FIG. 6; numbers in parentheses are negative), the following variants displayed a significantly increased PI (i.e., significantly greater than 0): D241N, G234D, F418M, T246S, T255R, T255P, T255I, T255V, Y247D, T255K, P194V, G340T, Y492A, S398F, E334A, Y370F, N49A, and S248K.

As shown in FIG. 6 and Table 3, the following variants displayed a significantly reduced PI (i.e., significantly less than 0): P258L, N200R, N49V, and F338E.

TABLE 3
CBH1 Variants Having Significant ΔPI values in daCS Assay

| Variant | ΔPI value |
|---|---|
| D241N | 0.12 |
| G234D | 0.11 |
| F418M | 0.063 |
| T246S | 0.049 |
| T255R | 0.035 |
| T255P | 0.031 |
| T255I | 0.029 |
| T255V | 0.025 |
| P194V | 0.025 |
| T255K | 0.020 |
| Y247D | 0.020 |
| Y492A | (0.020)* |
| Y370F | (0.023) |
| S398F | (0.024) |
| E334A | (0.024) |
| N49A | (0.043) |
| S248K | (0.046) |
| F338E | (0.060) |
| INTERCEPT | (0.062) |
| N49V | (0.068) |
| P258L | (0.078) |
| N200R | (0.081) |
| P194L | (0.29) |

*Numbers in parentheses are negative.

### Example 6

### CBH1 Variants with Significant Benefit in PASC PI Assay

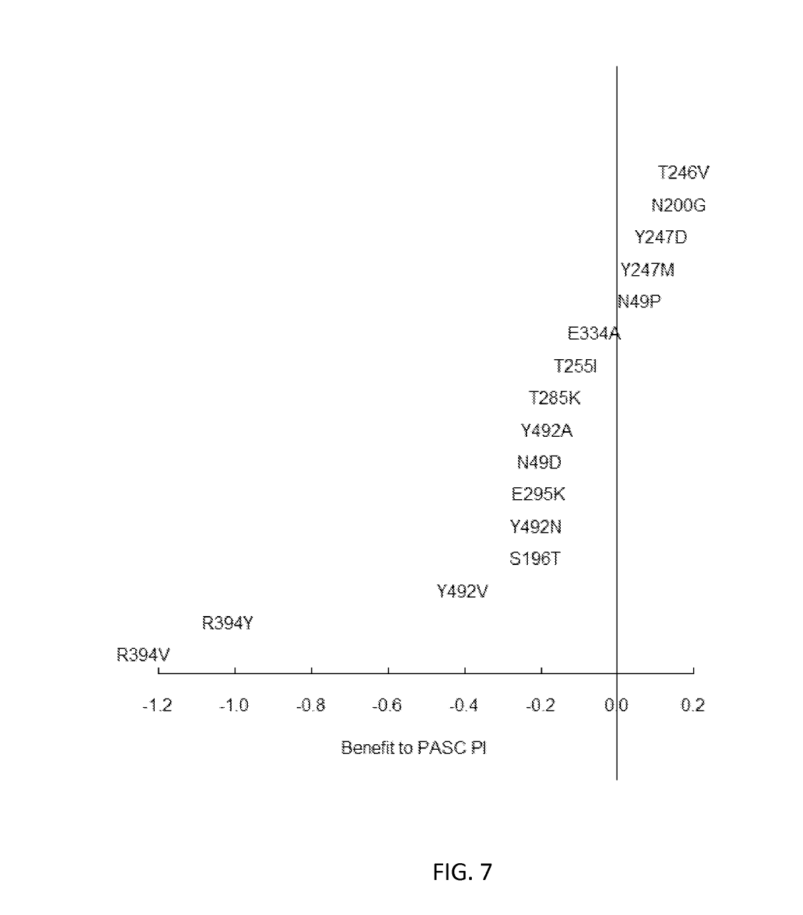

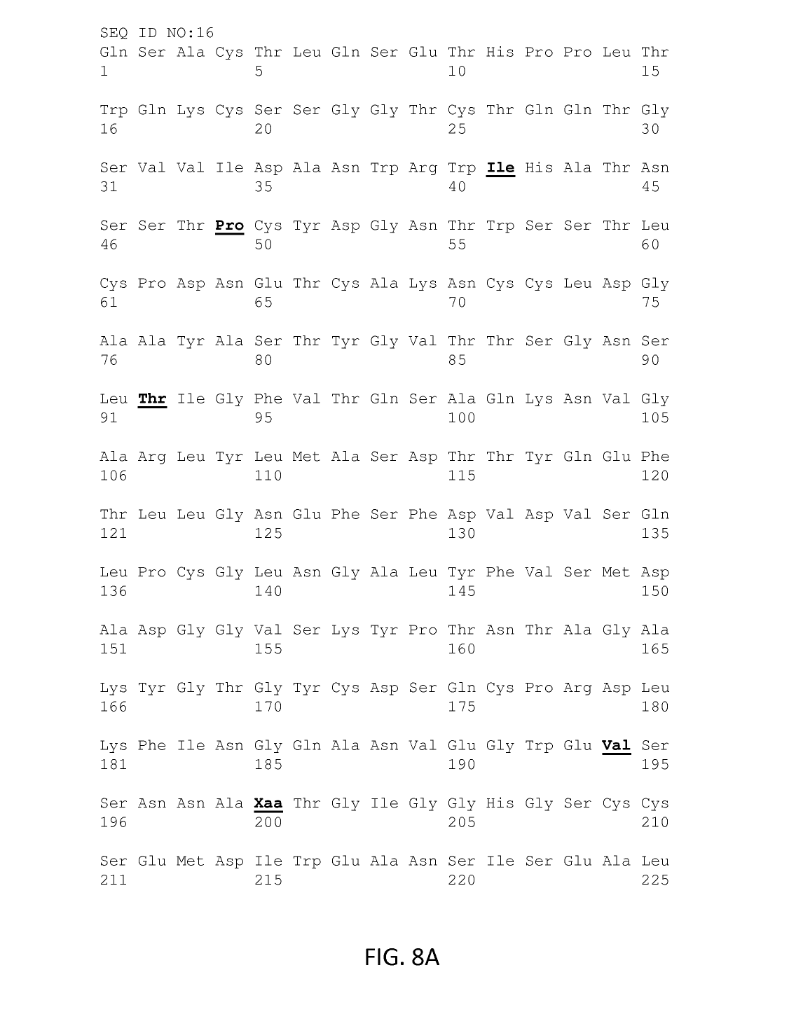

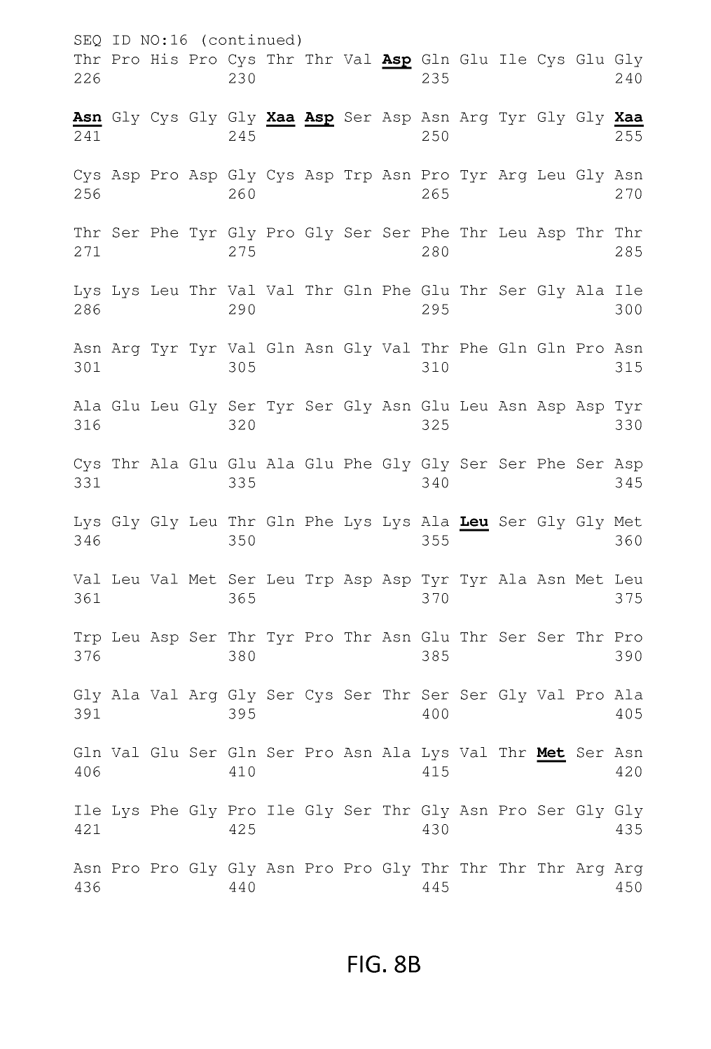

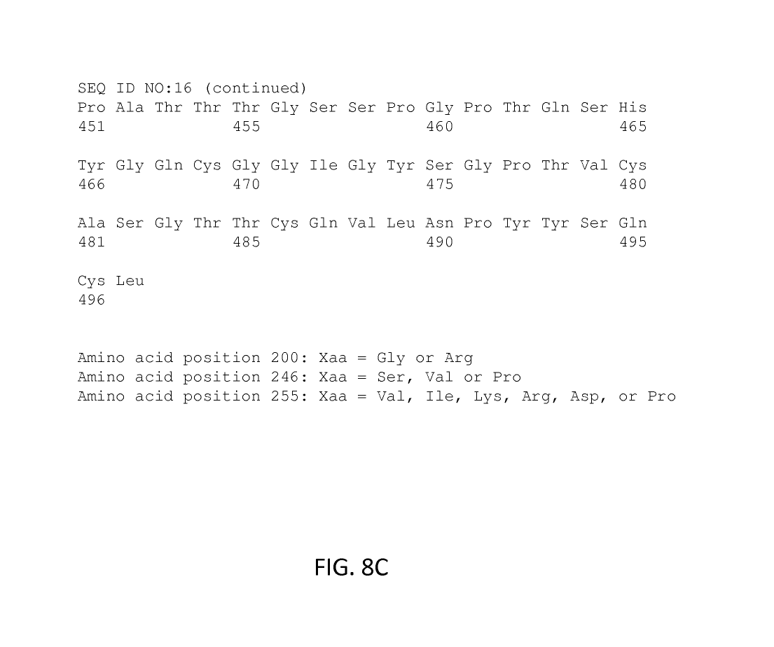

The performance index (PI) was determined for individually substituted CBH1 variants in one or more of the PASO assays as described above in D (D1 to D3) and analyzed to determine whether the PI as compared to the wild type CBH1 enzyme was significantly reduced or significantly increased (significance to 0.1, or 90%). The substitutions that display significant changes in PI are shown in the graph in FIG. 7 and in Table 4. In FIG. 7, the change in PI (or ΔPI value) is on the X axis (labeled “Benefit to PASO PI”) with each specific variant having significant change in PI shown at its approximate ΔPI value. The “intercept” value indicates the model's prediction of change in PI for a molecule with no substitutions (i.e., wild type CBH1). (In this model, the intercept was not significantly different than 0 and thus does not appear on the graph.)

As shown in FIG. 7 and Table 4 (which shows the ΔPI value for each CBH1 variant in FIG. 7; numbers in parentheses are negative), the following variants displayed a significantly increased PI (i.e., significantly greater than 0): T246V, N200G, Y247D, Y247M and N49P.

As shown in FIG. 7 and Table 4, the following variants displayed a significantly reduced PI (i.e., significantly less than 0): E334A, T255I, T285K, Y492A, N49D, E295K, Y492N, S196T, Y492V, R394Y, and R394V.

TABLE 4
CBH1 Variants Having Significant ΔPI values in PASC Assay

| Variant | ΔPI value |
|---|---|
| T246V | 0.18 |
| N200G | 0.16 |
| Y247D | 0.12 |
| Y247M | 0.081 |
| N49P | 0.060 |
| E334A | (0.057) |
| T255I | (0.11) |
| T285K | (0.16) |
| Y492A | (0.18) |
| N49D | (0.20) |
| E295K | (0.20) |
| Y492N | (0.21) |
| S196T | (0.21) |
| Y492V | (0.40) |
| R394Y | (1.0) |
| R394V | (1.2) |

* Numbers in parentheses are negative.

### Example 7

### Summary of Representative Data

Table 5 below shows the performance of each CBH1 variant having a beneficial effect in at least one assay in the whPCS, daCS, PASO and Tm assays. The number of “+” or “−” signs indicates the relative magnitude of the effect of the CBH1 variation on performance of the CBH1 enzyme in the indicated assay (based on values shown in Tables 1 to 4 above). The variants are also grouped into four Groups according to their performance characteristics. Group 1: benefit to whPCS and daCS; Group 2: benefit to daCS; Group 3: benefit to PASO; and Group 4: benefit to Tm. The variants in Group 5 are those that show a performance benefit in at least one assay and that find use in combination with other CBH1 variants. It is noted that any combination of the variants in Table 5 is contemplated (as described elsewhere herein).

TABLE 5
Summary of Properties of Representative CBH1 Variants

| Variant | whPCS (ΔPI) | daCS (ΔPI) | PASC (ΔPI) | ΔTm | Group |
|---|---|---|---|---|---|
| F418M | + | ++ | (ns) | (ns) | 1 |
| T246S | + | + | (ns) | (ns) | 1 |
| T255V | + | + | (ns) | (ns) | 1 |
| D241N | (ns) | +++ | (ns) | (ns) | 2 |
| G234D | (ns) | +++ | (ns) | (ns) | 2 |
| P194V | (ns) | + | (ns) | (ns) | 2 |
| T255I | (ns) | + | − − − | (ns) | 2 |
| T255K | (ns) | + | (ns) | (ns) | 2 |
| T255R | (ns) | + | (ns) | (ns) | 2 |
| N200G | − | (ns) | ++++ | (ns) | 3 |
| N49P | − * | (ns) | ++ | (ns) | 3 |
| T246V | − * | (ns) | ++++ | ++ | 3 |
| Y247D | − * | + | +++ | (ns) | 3 |
| N200R | − − | − − | (ns) | ++ | 4 |
| T246P | − * | (ns) | (ns) | ++ | 4 |
| T255D | − * | (ns) | (ns) | ++ | 4 |
| T356L | (ns) | (ns) | (ns) | ++ | 4 |
| S92T | ++++ | (ns) | (ns) | (ns) | 5 |
| T255P | (ns) | + | (ns) | +++ | 5 |
| T41I | (ns) | (ns) | (ns) | ++++ | 5 |

* The modeled ΔPI is greater than the modeled ΔPI for the intercept (i.e., wild type).
(ns) = not significantly different from 0.

As noted above, any combination of variants in Table 5 finds use in aspects of the present invention.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### REFERENCES

Altschul, S. F., et al., J. Mol. Biol. 215:403-410, 1990.
Altschul, S. F., et al., Nucleic Acids Res. 25:3389-3402, 1997.
Aro, N., et al., J. Biol. Chem., 10.1074/M003624200, Apr. 13, 2001.
Aubert, et al., Ed., p11 et seq., Academic Press, 1988.
Ausubel G. M., et al. CURRENT PROTOCOLS IN MOLECULAR BIOLOGY, John Wiley & Sons, New York, N.Y., 1993.
Baldwin, D., et al., Curr. Opin. Plant Biol. 2(2):96-103, 1999.
Baulcombe, D., Arch. Virol. Suppl. 15:189-201, 1999.
Bhikhabhai, R. et al., J. Appl. Biochem. 6:336, 1984.
Boer and Koivula, 2003, Eur. J. Biochem. 270: 841-848
Brumbauer, A. et al., Bioseparation 7:287-295, 1999.
Carter et al., Nucl. Acids Res. 13:4331, 1986.
Chen et al., Biochem. Biophys. Acta. 1121:54-60, 1992.
Coligan, J. E. et al., eds., CURRENT PROTOCOLS IN IMMUNOLOGY, 1991.
Collen, A., et al., Journal of Chromatography A 910:275-284, 2001.
Coughlan, et al., BIOCHEMISTRY AND GENETICS OF CELLULOSE DEGRADATION.
Cummings and Fowler, Curr. Genet. 29:227-233, 1996.
Dayhoff et al. in Atlas of Protein Sequence and Structure, Volume 5, Supplement 3, Chapter 22, pp. 345-352, 1978.
Deutscher, M. P., Methods Enzymol. 182:779-80, 1990.
Doolittle, R. F., OF URFS AND ORFs, University Science Books, C A, 1986.
Ellouz, S. et al., J. Chromatography 396:307, 1987.
Fields and Song, Nature 340:245-246, 1989.
Filho, et al. Can. J. Microbiol. 42:1-5, 1996.
Fliess, A., et al., Eur. J. Appl. Microbiol. Biotechnol. 17:314, 1983.
Freer, et al. J. Biol. Chem. 268:9337-9342, 1993.
Freshney, R. I., ed., ANIMAL CELL CULTURE, 1987.
Goyal, A. et al. Bioresource Technol. 36:37, 1991.
Halldorsdottir, S et al., Appl Microbiol Biotechnol. 49(3):277-84, 1998.
Hu et al., Mol Cell Biol. 11:5792-9, 1991.
Hemmpel, W. H. ITB Dyeing/Printing/Finishing 3:5-14, 1991.
Herr et al., Appl. Microbiol. Biotechnol. 5:29-36, 1978.
Jakobovits, A, et al., Ann N Y Acad Sci 764:525-35, 1995.
Jakobovits, A, Curr Opin Biotechnol 6(5):561-6, 1995.
Jones et al., Nature 321:522-525, 1986.
Kawaguchi, T et al., Gene 173(2):287-8, 1996.
Knowles, J. et al., TIBTECH 5, 255-261, 1987.
Kohler and Milstein, Nature 256:495, 1975.
Krishna, S. et al., Bioresource Tech. 77:193-196, 2001.
Kumar, A., et al., Textile Chemist and Colorist 29:37-42, 1997.
Lehtio, J. et al., FEMS Microbiology Letters 195:197-204, 2001.
Li and Ljungdahl Appl. Environ. Microbiol. 62:209-213, 1996.
Linder, M. and Teeri, T. T., Biotechnol. 57:15-28, 1997.
Medve, J. et al., J. Chromatography A 808:153, 1998.
Ohmiya et al., Biotechnol. Gen. Engineer. Rev. 14:365-414, 1997.
Ooi et al., Nucleic Acids Res. 18(19):5884, 1990.
Ortega et al., International Biodeterioration and Biodegradation 47:7-14, 2001.
Penttila et al., Yeast 3:175-185, 1987.
Penttila et al., Gene 63: 103-112, 1988.
Pere, J., et al., In Proc. Tappi Pulping Conf., Nashville, Tenn., 27-31, pp. 693-696, 1996.
Riechmann et al., Nature 332:323-327, 1988.
Rothstein et al., Gene 55:353-356, 1987.
Saarilahti et al., Gene 90:9-14, 1990.
Sakamoto et al., Curr. Genet. 27:435-439, 1995.
Saloheimo M, et al., Gene 63:11-22, 1988.
Sambrook et al., MOLECULAR CLONING: A LABORATORY MANUAL (Second Edition), Cold Spring Harbor Press, Plainview, N.Y., 1989.
Schulein, Methods Enzymol., 160, 25, pages 234 et seq, 1988.
Scopes, Methods Enzymol. 90 Pt E:479-90, 1982.
Spilliaert R, et al., Eur J Biochem. 224(3):923-30, 1994.
Stahlberg, J. et al., Bio/Technol. 9:286-290, 1991.
Stahlberg et al., 1996, J. Mol. Biol. 264: 337-349
Strathern et al., eds. (1981) The Molecular Biology of the Yeast Saccharomyces.
Suurnakki, A. et al., Cellulose 7:189-209, 2000.
Te'o, J. et al., FEMS Microbiology Letters 190:13-19, 2000.
Tilbeurgh, H. et al., FEBS Lett. 16:215, 1984.
Timberlake et al., Cell 1:29-37, 1981.
Tomaz, C. and Queiroz, J., J. Chromatography A 865:123-128, 1999.
Tomme, P. et al., Eur. J. Biochem. 170:575-581, 1988.
Tormo, J. et al., EMBO J. 15:5739-5751, 1996.
Tyndall, R. M., Textile Chemist and Colorist 24:23-26, 1992.
Van Rensburg et al., Yeast 14:67-76, 1998.
Van Tilbeurgh, H. et al., FEBS Lett. 204:223-227, 1986.
Verhoeyen et al., Science 239:1534-1536, 1988.
Warrington, et al., Genomics 13:803-808, 1992.
Wells et al., Gene 34:315, 1985.
Wells et al., Philos. Trans. R. Soc. London SerA 317:415, 1986.
Wood, Biochem. Soc. Trans., 13, pp. 407-410, 1985.
Wood et al., METHODS IN ENZYMOLOGY, 160, 25, p. 87 et seq., Academic Press, New York, 1988.
Zoller et al., Nucl. Acids Res. 10:6487, 1987.

1. (canceled)

2. An isolated variant of a parent cellobiohydrolase (CBH) enzyme, wherein said variant has cellulase activity, has at least 80% sequence identity to SEQ ID NO:3, and wherein said variant comprises an amino acid substitution selected from the group consisting of: Y247D, N49P, T246V, N200G, and combinations thereof, wherein the position of each amino acid substitution corresponds to SEQ ID NO:3.

3. The isolated variant of claim 2, wherein said variant comprises a Y247D substitution.

4. The isolated variant of claim 2, wherein said variant comprises a N49P substitution.

5. The isolated variant of claim 2, wherein said variant comprises a T246V substitution.

6. The isolated variant of claim 2, wherein said variant comprises a N200G substitution.

7. The isolated variant of claim 2, wherein said variant further comprises an amino acid substitution selected from the group consisting of: T246S and T246P.

8. The isolated variant of claim 2, wherein said variant further comprises an N200R amino acid substitution.

9. The isolated variant of claim 2, wherein said variant further comprises an amino acid substitution selected from the group consisting of: F418M, T255D or T255I or T255K or T255P or T255R or T255V, D241N, G234D, P194V, T356L, S92T, T41I, and combinations thereof.

10. The isolated variant of claim 2, wherein said parent CBH polypeptide is a fungal cellobiohydrolase 1 (CBH1).

11. The isolated variant of claim 10, wherein said fungal CBH1 is from Hypocrea jecorina, Hypocrea schweinitzii, Hypocrea orientalis, Trichoderma pseudokoningii, Trichoderma konilangbra, Trichoderma citrinoviride, Trichoderma harzanium, Aspergillus aculeatus, Aspergillus niger; Penicillium janthinellum, Humicola grisea, Scytalidium thermophilum, or Podospora anderina.

12-15. (canceled)

16. A host cell comprising a polynucleotide sequence encoding a variant of a parent CBH polypeptide according to claim 2.

17. The host cell of claim 16, wherein said host cell is a fungal cell or a bacterial cell.

18. The host cell of claim 17, wherein said host cell is selected from the group consisting of:
a filamentous fungal cell selected from the group consisting of: Trichoderma reesei, Trichoderma longibrachiatum, Trichoderma viride, Trichoderma koningii, Trichoderma harzianum, Penicillium, Humicola, Humicola insolens, Humicola grisea, Chrysosporium, Chrysosporium lucknowense, Myceliophthora thermophilia, Gliocladium, Aspergillus, Fusarium, Neurospora, Hypocrea, Emericella, Aspergillus niger, Aspergillus awamori, Aspergillus aculeatus, and Aspergillus nidulans;
a yeast cell selected from the group consisting of: Saccharomyces cervisiae, Schizzosaccharomyces pombe, Schwanniomyces occidentalis, Kluveromyces lactus, Candida utilis, Candida albicans, Pichia stipitis, Pichia pastoris, Yarrowia lipolytica, Hansenula polymorpha, Phaffia rhodozyma, Arxula adeninivorans, Debaryomyces hansenii, and Debaryomyces polymorphus; and
a Zymomonas mobilis bacterial cell.

19. The host cell of claim 17, wherein said host cell expresses the variant of a parent CBH polypeptide.

20-23. (canceled)

24. A method for hydrolyzing a cellulosic substrate, comprising:
contacting said substrate with an isolated variant of a parent cellobiohydrolase (CBH) enzyme, wherein said variant has cellulase activity, has at least 80% sequence identity to SEQ ID NO:3, and wherein said variant comprises an amino acid substitution selected from the group consisting of: Y247D, N49P, T246V, N200G, and combinations thereof, wherein the position of each amino acid substitution corresponds to SEQ ID NO:3.

25. The method of claim 24, wherein said cellulosic substrate is of a lignocellulosic biomass is selected from the group consisting of grass, switch grass, cord grass, rye grass, reed canary grass, miscanthus, sugar-processing residues, sugarcane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, wood pulp, recycled wood pulp fiber, paper sludge, sawdust, hardwood, softwood, and combinations thereof.

26-33. (canceled)

34. The method of claim 24 wherein the variant is produced by co-expression with one or more other cellulases or hemicellulases.

35. The host cell of claim 17 wherein said host cell has had one or more cellulase genes deleted.


SEQUENCE LISTING (16 sequences)

SEQ ID NO: 1 - DNA, 1542 bp, Hypocrea jecorina
  misc_feature: includes the wild type nucleotide sequence encoding the mature form of H. jecorina cbh1 adjacent to a sequence encoding the CBH1 signal peptide
atgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgagacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgattactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctg

SEQ ID NO: 2 - protein, 514 aa, Hypocrea jecorina
  misc_feature: sequence of the H. jecorina CBH1 full length polypeptide containing the CBH1 signal peptide
MYRKLAVISAFLATARAQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNPPGTTTTRRPATTTGSSPGPTQSHYGQCGGIGYSGPTVCASGTTCQVLNPYYSQCL

SEQ ID NO: 3 - protein, 497 aa, Hypocrea jecorina [sequence omitted]

SEQ ID NO: 4 - protein, 497 aa, Hypocrea orientalis
QSACTLQTETHPSLTWQKCSSGGTCTQQTGSVVIDANWRWTHATNSSTNCYDGNTWSSTLCPDNETCAKNCCLDGAAYASTYGVTTSADSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICDGDGCGGTYSNDRYGGTCDPDGCDWNPYRLGNTSFYGPGSSFTLDTTKKLTVVTQFETSGAINRYYVQNGVTYQQPNAELGSYSGNELNDDYCTAEESEFGGSSFSDKGGLTQFKKATSGGMVLVMSLWDDYYANMLWLDSTYPTNETSSTPGAVRGSCSTSSGVPAQLESQSPNAKVVYSNIKFGPIGSTGNPSGGNPPGGNPPGTTTTRRPATTTGSSPGPTQTHYGQCGGIGYSGPTVCASGTTCQVLNPYYSQCL

SEQ ID NO: 5 - protein, 497 aa, Hypocrea schweinitzii
QSACTLQTETHPSLTWQKCSSGGTCTQQTGSVVIDANWRWTHATNSSTNCYDGNTWSSTLCPDNETCAKNCCLDGAAYASTYGVTTSADSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTNVGQEICDGDGCGGTYSNDRYGGTCDPDGCDWNPYRLGNTSFYGPGSSFTLDTTKKLTVVTQFETSGAINRYYVQNGVTYQQPNAELGSYSGNELNDAYCTAEESEFGGSSFSDKGGLTQFKKATSGGMVLVMSLWDDYYANMLWLDSTYPTNETSSTPGAVRGSCSTSSGVPAQLESQSANAKVVYSNIKFGPIGSTGNPSGGNPPGGNPPGTTTTRRPATTTGSSPGPTQTHYGQCGGIGYSGPTICASGTTCQVLNPYYSQCL

SEQ ID NO: 6 - protein, 497 aa, Trichoderma citrinoviride
  misc_feature (217)..(217): Xaa can be any naturally occurring amino acid
QSACTLQTETHPSLTWQKCSSGGTCTQQTGSVVIDANWRWTHATNSSTNCYDGNTWSSTLCPDNETCAKNCCLDGAAYASTYGVTTSADSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWXANSISEALTPHPCTTVGQEICEGDGCGGTYSNDRYGGTCDPDGCDWNPYRLGNTSFYGPGSSFTLDTTKKLTVVTQFETSGAINRYYVQNGVTYKQPNAELGSYSGNELNDDYCTAEESEFGGSSFSDKGGLTQFKKATSGGMVLVMSLWDDYYANMLWLDSTYPTNETSSTPGAVRGSCSTSSGVPAQLESQSPNAKVVYSNIKFGPIGSTGNPSGGNPPGGNPPGTTTTRRPATTTGSSPGPTQTHYGQCGGIGYSGPTVCASGTTCQVLNEYYSQCL

SEQ ID NO: 7 - protein, 498 aa, Trichoderma pseudokoningii
QSACTLQTETHPPLTWQKCSSGGTCTQQTGSVVIDANWRWTHATNSSTNCYDGNTWSSTLCPDNETCAKNCCLDGAAYASTYGVTTSADSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGEANVEGWEPFSNNANTGIGGHGSCCSEMDIWEANSISEALTPHPCTTVGQEICDGDSCGGTYSGDRYGGTCDPDGCDWNPYRLGNTSFYGPGSSFALDTTKKLTVVTQFETSGAINRYYVQNGVTFQQPNAELGSYSGNSLDDDYCAAEEAEFGGSSFSDKGGLTQFKKATSGGMVLVMSLWDDYYANMLWLDSTYPTNETSSTPGAVRGSCSTSSGVPAQLESQSSNAKVVYSNIKFGPIGSTGNSSGGSPPGGGNPPGTTTTRRPATSTGSSPGPTQTHYGQCGGIGYSGPTVCASGSTCQVLNPYYSQCL

SEQ ID NO: 8 - protein, 498 aa, Trichoderma konilangbra
  misc_feature (273)..(273): Xaa can be any naturally occurring amino acid
QSACTIQAETHPPLTWQKCSSGGSCTSQTGSVVIDANWRWTHATNSTTNCYDGNTWSSSLCPDNESCAKNCCLDGAAYASTYGVTTSADSLSIGFVTQSQQKNVGARLYLMASDTTYQEFTLLGNEFSFDVDVSQLPCGLNGALYFVSMDADGGVSKYPSNTAGAKYGTGYCDSQCPRDLKFINGEANVEGWEPASNNANTGIGGHGSCCSEMDIWEANSISEALTPHPCTTVGQAICDGDGCGGTYSDDRYGGTCDPDGCDWNPYRLGNTSXYGPGSSFTLDTTKKMTVVTQFATSGAINRYYVQNGVTFQQPNAELGSYSGNTLNDAYCAAEEAEFGGSSFSDKGGLTQFKQATSGGMVLVMSLWDDYYANMLWLDSIYPTNETSSTPGAARGSCSTSSGVPAQLESQSTNAKVVFSNIKFGPIGSTGNSSGGNPPGGGNPPGTTTTRRPATTTGSSPGPTQTHYGQCGGIGYSGPTVCASGSTCQVLNPYYSQCL

SEQ ID NO: 9 - protein, 488 aa, Trichoderma harzanium
QQVCTQQAETHPPLTWQKCTASGCTPQQGSVVLDANWRWTHDTKSTTNCYDGNTWSSTLCPDDATCAKNCCLDGANYSGTYGVTTSGDALTLQFVTASNVGSRLYLMANDSTYQEFTLSGNEFSFDVDVSQLPCGLNGALYFVSMDADGGQSKYPGNAAGAKYGTGYCDSQCPRDLKFINGQANVEGWEPSSNNANTGVGGHGSCCSEMDIWEANSISEALTPHPCETVGQTMCSGDSCGGTYSNDRYGGTCDPDGCDWNPYRLGNTSFYGPGSSFALDTTKKLTVVTQFATDGSISRYYVQNGVKFQQPNAQVGSYSGNTINTDYCAAEQTAFGGTSFTDKGGLAQINKAFQGGMVLVMSLWDDYAVNMLWLDSTYPTNATASTPGAKRGSCSTSSGVPAQVEAQSPNSKVIYSNIRFGPIGSTGGNTGSNPPGTSTTRAPPSSTGSSPTATQTHYGQCGGTGWTGPTRCASGYTCQVLNPFYSQCL

SEQ ID NO: 10 - protein, 518 aa, Aspergillus aculeatus
QQVGTYTAETHPSLTWQTCSGSGSCTTTSGSVVIDANWRWVHEVGGYTNCYSGNTWDSSICSTDTTCASECALEGATYESTYGVTTSGSSLRLNFVTTASQKNIGSRLYLLADDSTYETFKLFNREFTFDVDVSNLPCGLNGALYFVSMDADGGVSRFPTNKAGAKYGTGYCDSQCPRDLKFIDGQANIEGWEPSSTDVNAGTGNHGSCCPEMDIWEANSISSAFTAHPCDSVQQTMCTGDTCGGTYSDTTDRYSGTCDPDGCDFNPYRFGNTNFYGPGKTVDNSKPFTVVTQFITHDGTDTGTLTEIRRLYVQNGVVIGNGPSTYTAASGNSITESFCKAEKTLFGDTNVFETHGGLSAMGDALGDGMVLVLSLWDDHAADMLWLDSDYPTTSCASSPGVARGTCPTTTGNATYVEANYPNSYVTYSNIKFGTLNSTYSGTSSGGSSSSSTTLTTKASTSTTSSKTTTTTSKTSTTSSSSTNVAQLYGQCGGQGWTGPTTCASGTCTKQNDYYSQCL

SEQ ID NO: 11 - protein, 515 aa, Aspergillus niger
QQVGTYTTETHPSLTWQTCTSSGSCTTNDGAVVIDANWRWVHSTSSSTNCYTGNEWDTSICTDDVTCAANCALDGATYEATYGVTTSGDELRLNFVTQGSSKNIGSRLYLMSDDSTYEMFKLLGQEFTFDVDVSNLPCGLNGALYFVAMDADGGTSEYSGNKAGAKYGTGYCDSQCPRDLKFINGEANCDGWEPSSNNVNTGVGDHGSCCPEMDIWEANSISNAFTAHPCDSVSQTMCDGDSCGGTYSASGDRYSGTCDPDGCDYNPYRLGNTDFYGPGLTVDTNSPFTVVTQFITDDGTSSGTLTEIKRLYVQNGEVIANGASTYSSVNGSSITSDFCESEKTLFGDENVFDTHGGLAGMGEAMANGMVLVLSLWDDYAANMLWLDSDYPVNSSASTPGVARGTCSTDSGVPATVEADSPNAYVTYSNIKFGPIGSTYSSGSSSGSGSSSSSSSTTTKATSTTLKTTTTTSSGSSSTSAAQAYGQCGGQSWTGPTTCVSGYTCTYQNAYYSQCL

SEQ ID NO: 12 - protein, 512 aa, Penicillium janthinellum
  misc_feature (23)..(23): Xaa can be any naturally occurring amino acid
QQVGTLTAETHPALTWSKCTAGXCSQVSGSVVIDANWPXVHSTSGSTNCYTGNTWDATLCPDDVTCAANCAVDGARRQHLRVTTSGNSLRINFVTTASQKNIGSRLYLLENDTTYQKFNLLNQEFTFDVDVSNLPCGLNGALYFVDMDADGGMAKYPTNKAGAKYGTGYCDSQCPRDLKFINGQANVDGWTPSKNDVNSGIGNHGSCCAEMDIWEANSISNAVTPHPCDTPSQTMCTGQRCGGTYSTDRYGGTCDPDGCDFNPYRMGVTNFYGPGETIDTKSPFTVVTQFLTNDGTSTGTLSEIKRFYVQGGKVIGNPQSTIVGVSGNSITDSWCNAQKSAFGDTNEFSKHGGMAGMGAGLADGMVLVMSLWDDHASDMLWLDSTYPTNATSTTPGAKRGTCDISRRPNTVESTYPNAYVIYSNIKTGPLNSTFTGGTTSSSSTTTTTSKSTSTSSSSKTTTTVTTTTTSSGSSGTGARDWAQCGGNGWTGPTTCVSPYTCTKQNDWYSQCL

SEQ ID NO: 13 - protein, 507 aa, Humicola grisea
QQACSLTTERHPSLSWKKCTAGGQCQTVQASITLDSNWRWTHQVSGSTNCYTGNKWDTSICTDAKSCAQNCCVDGADYTSTYGITTNGDSLSLKFVTKGQHSTNVGSRTYLMDGEDKYQTFELLGNEFTFDVDVSNIGCGLNGALYFVSMDADGGLSRYPGNKAGAKYGTGYCDAQCPRDIKFINGEANIEGWTGSTNDPNAGAGRYGTCCSEMDIWEANNMATAFTPHPCTIIGQSRCEGDSCGGTYSNERYAGVCDPDGCDFNSYRQGNKTFYGKGMTVDTTKKITVVTQFLKDANGDLGEIKRFYVQDGKIIPNSESTIPGVEGNSITQDWCDRQKVAFGDIDDFNRKGGMKQMGKALAGPMVLVMSIWDDHASNMLWLDSTFPVDAAGKPGAERGACPTTSGVPAEVEAEAPNSNVVFSNIRFGPIGSTVAGLPGAGNGGNNGGNPPPPTTTTSSAPATTTTASAGPKAGRWQQCGGIGFTGPTQCEEPYTCTKLNDWYSQCL

SEQ ID NO: 14 - protein, 515 aa, Scytalidium thermophilum
QQACSLTTERHPSLSWKKCTAGGQCQTVQASITLDSNWRWTHQVSGSTNCYTGNEWDSSICTDAKSCAQNCCVDGADYTSTYGITTNGDSLSLKFVTKGQYSTNVGSRTYLMDGEDKYQTFELLGNEFTFDVDVSNIGCGLNGALYFVSMDADGGLSRYPGNKAGAKYGTGYCDAQCPRDIKFINGEANIEGWTGSTNDPNAGAGRYGTCCSEMDIWEANNMATAFTPHPCTIIGQSRCEGDSCGGTYSNDRYAGVCDPDGCDFNAYRQGNKTFYGKGMTVDTTKKLTVVTQFLKDANGDLGEIKRFYVQDGKIIPNSESTIPGVEGNSITQDWCDRQKVAFGDIDDFNRKGGMKQMGKALAGPMVLVMSIWDDHASNMLWLDSTFPVDAAGKPGAERGACPTTSGVPAEVEAEAPNSNVVFSNIRFGPIGSTVAGLPSDGGNNGGNTTVQPPPSTTTTSASSSTTSAPATTTTASAGPKAGRWQQCGGIGFTGPTQCEEPYTCTKLNDWYSQCL

SEQ ID NO: 15 - protein, 509 aa, Podospora anderina
QQVCSLTPESHPPLTWQRCSAGGSCTNVAGSVTLDSNWRWTHTLQGSTNCYSGNEWDTSICTTGTKCAQNCCVEGAEYAATYGITTSGNQLNLKFVTEGKYSTNVGSRTYLMENATKYQGFNLLGNEFTFDVDVSNIGCGLNGALYFVSMDLDGGLAKYSGNKAGAKYGTGYCDAQCPRDIKFINGEANIEGWNPSTNDVNAGAGRYGTCCSEMDIWEANNMATAYTPHSCTILDQSRCEGESCGGTYSSDRYGGVCDPDGCDFNSYRMGNKEFYGKGKTVDTTKKMTVVTQFLKNAAGELSEIKRFYVQNGVVIPNSVSSIPGVPNQNSITQDWCDAQKIAFGDPDDNTAKGGLRQMGLALDKPMVLVMSIWNDHAAHMLWLDSTYPVDAAGRPGAERGACPTTSGVPSEVEAEAPNSNVAFSNIKFGPIGSTFNSGSTNPNPISSSTATTPTSTRVSSTSTAAQTPTSAPGGTVPRWGQCGGQGYTGPTQCVAPYTCVVSNQWYSQC

SEQ ID NO: 16 - protein, 497 aa, Hypocrea jecorina
  misc_feature: CBH1 amino acid sequence from H. jecorina
QSACTLQSETHPPLTWQKCSSGGTCTQQTGSVVIDANWRWIHATNSSTPCYDGNTWSSTLCPDNETCAKNCCLDGAAYASTYGVTTSGNSLTIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEVSSNNAXTGIGGHGSCCSEMDIWEANSISEALTPHPCTTVDQEICEGNGCGGXDSDNRYGGXCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKALSGGMVLVMSLWDDYYANMLWLDSTYPTNETSSTPGAVRGSCSTSSGVPAQVESQSPNAKVTMSNIKFGPIGSTGNPSGGNPPGGNPPGTTTTRRPATTTGSSPGPTQSHYGQCGGIGYSGPTVCASGTTCQVLNPYYSQCL